(19) United States
(12) Patent Application Publication
(10) Pub. No.: US 2021/0346293 A1
(43) Pub. Date: Nov. 11, 2021

(54) Lipid Vesicle for Oral Drug Delivery

(71) Applicant: Hoffmann-La Roche Inc., Little Falls, NJ (US)

(72) Inventors: Philip Grossen, Basel (CH); Michael Keller, Basel (CH)

(21) Appl. No.: 17/384,573

(22) Filed: Jul. 23, 2021

Related U.S. Application Data
(63) Continuation of application No. PCT/EP2020/051695, filed on Jan. 24, 2020.

(30) Foreign Application Priority Data
Jan. 25, 2019 (EP) 19153729.9

(51) Int. Cl. A61K 9/127; A61K 48/00; C12N 15/113

(52) U.S. Cl. CPC A61K 9/127; C12N 2310/11; C12N 15/113; A61K 48/0008

(57) ABSTRACT

The present invention relates to a lipid vesicle carrying a nucleic acid molecule for use as a medicament, wherein the lipid vesicle has a hydrodynamic diameter Dₕ of less than 300 nm. The lipid vesicle is for oral administration, and the nucleic acid molecule is delivered to one or more of the target tissues selected from the group consisting of the central nervous system, spleen, gastrointestinal tract, liver and T-cells.

### FIELD OF INVENTION

The present invention relates to a lipid vesicle carrying a nucleic acid molecule for use as a medicament, wherein the lipid vesicle has a hydrodynamic diameter Dₕ of less than 300 nm.

The lipid vesicle is for oral administration, and the nucleic acid molecule is delivered to one or more of the target tissues selected from the group consisting of the central nervous system, spleen, gastrointestinal tract, liver and T-cells.

### BACKGROUND

The delivery of drugs to specific cells and tissues is influenced by many factors, such as the stability of the drug, the metabolism of the drug before reaching the target tissue, the ability of the drug to cross cell membranes, tight junctions between cells or the blood-brain barrier. Therapies of diseases with large molecular drugs (e.g. nucleic acids, peptides, and proteins) are currently limited to parenteral administration which generally results in systemic distribution of the drug overtime. This can mainly be attributed to the chemical and physical properties of these drugs not meeting Lipinski's rule of five as well as potential low stability of many drugs at harsh conditions of the gastro-intestinal tract (GIT). Several strategies to orally administer such drugs were described recently, e.g. the use of milk exosomes (mExo) for delivery of micro RNAs (miRNA). Exosomes are extracellular vesicles secreted by all types of cells. In a recent publication Manca et al. 2018 Scientific Reports 8:11321 the distribution of milk exosomes comprising synthetic miRNAs after oral administration was analyzed. According to Manca, distinct miRNA cargos have unique tissue distribution patterns ( ). Further, Munagala et al. 2016 Cancer Lett. 371(1): 48-61 describes the isolation of exosomes from bovine milk and the encapsulation of a chemotherapeutic agent into the isolated exosomes.

Whereas initial results look promising, technical limitations such as isolation and purification of milk exosomes, batch-to-batch variabilities, microbial contaminations, and efficient drug loading need to be solved before such systems can be applied on a commercial scale (see. e.g. Somiya et al. 2018 J Extracell Vesicles. 7(1):1440132).

Alternative systems that offer technical developmentability and that can be produced in a robust and reproducible manner are of big need.

Lu et al. 2018 Int J Pharm. 550(1-2):100-113 describes the production of exosome-mimicking vesicles and the comparison of produced vesicles with conventional liposomes ( ). It was shown that the exosome-mimicking vesicles were capable of delivering VEGF siRNA to A549 and HUVEC cells. However, no in vivo experiments were carried out.

Oral delivery of therapeutic nucleic acid molecules in exosome-mimicking has to our knowledge never been shown to result in target modulation in the CNS, gastrointestinal tract, spleen or T-cells.

### OBJECTIVE OF THE INVENTION

The inventors have shown in the studies underlying the present invention that oral administration of lipid vesicles carrying a nucleic acid molecule, such as a single-stranded antisense oligonucleotide allows for modulation of a target nucleic acid in the central nervous system (e.g. the brain), the spleen, the gastrointestinal tract, the liver and/or in T-cells (see Examples section). The lipid vesicles have a hydrodynamic diameter Dₕ of less than 300 nm and mimic the properties of naturally occurring extracellular vesicles. They can be easily produced in large amounts without requiring the time-consuming and costly isolation from milk. In addition, they lack potential contamination from live source isolated exosomes such as milk, cell cultures and natural body fluids.

The lipid vesicles comprising one or more nucleic acid molecules, when administered orally, are capable of delivering the said nucleic acid molecules to one or more of the target tissues selected from the group consisting of the central nervous system, spleen, gastrointestinal tract, liver and T-cells resulting in modulation of a target nucleic acid in the tissue. The findings of the present invention are particularly useful in the treatment of a range of diseases, such as diseases which are associated with abnormal expression, such as increased expression, reduced expression or undesired suppression, splice-switching errors or mutational errors, of a target nucleic in the central nervous system (e.g. the brain), the spleen, the gastrointestinal tract, the liver and/or in T-cells.

### BRIEF DESCRIPTION OF FIGURES

FIG. 1: Summary of lipid components in lipid vesicles F1-F34 tested in the present application as well as their assessed stability for oral delivery based on size of the unloaded vesicle and size change upon incubation in simulated gastric fluid (SGF) or simulated fasted state intestinal fluid (FaSSIF). ++=stable vesicle with Dh between 60 and 180 nm and % change in Dh below 60% in SGF and below 25% in FaSSIF; +=stable vesicle with Dh between 50 and 300 nm and % change in Dh below 90% in SGF and below 40% in FaSSIF; −=Dh before or after incubation in SGF and FaSSIF above 300 or % change in Dh above 90% in SGF and above 40% in FaSSIF. The lipid components are abbreviated as follows Chol=cholesterol, SM=sphingomyelin, PC=phosphatidylcholine, DSPC=1,2-distearoyl-sn-glycero-3-phosphocholine, DLPC=1,2-dilauroyl-sn-glycero-3-phosphocholine, DOPC=dioleoyl-phosphatidylcholine, PE=phosphatidylethanolamine, PS=phosphatidylserine, DAG=diacylglycerol, TAG=triacylglycerol, PI=phosphatidylinositol, LBPA=lysobisphosphatidic acid.

### STATEMENT OF THE INVENTION

The invention provides a lipid vesicle carrying a nucleic acid molecule for use as a medicament, wherein said lipid vesicle is administered orally, wherein said nucleic acid molecule is for delivery to one or more of the target tissues selected from the group consisting of the central nervous system, spleen, gastrointestinal tract, liver and T-cells. Preferably, the lipid vesicle has a hydrodynamic diameter Dₕ of less than 300 nm, measured by dynamic light scattering (DLS), as described in more detail herein.

Advantageously, the lipid vesicle is stable in gastric fluid at a temperature of 20° C. for up to 5 h.

In an embodiment, the lipid vesicle has a hydrodynamic diameter of less than 180 nm, measured according to DLS, in particular a diameter in the range from 60 to 180 nm.

In an embodiment, the lipid vesicle comprises at least one phosphatidylethanolamine in an amount in the range from 10 to 70 mole percent (mol %), and at least one sphingomyelin in an amount in the range from 2 to 45 mol %, preferably 4 to 15 mol %, based on the total amount [mol] of the unloaded lipid vesicle as such, wherein the lipid vesicle optionally comprises at least one diacylglycerol and/or at least one triacylglycerol and/or at least one phosphatidylinositol, wherein the total amount of the sum of diacylglycerols and triacylglycerols and phosphatidylinositols is less than 15 mol %, based on the total amount [mol] of the unloaded lipid vesicle as such. In a further embodiment the lipid vesicle comprises less than 5 mol % of diacylglycerols and less than 5 mol % of triacylglycerols and less than 5 mol % of phosphatidylinositols, based on the total amount [mol] of the unloaded lipid vesicle as such.

In an embodiment, the lipid vesicle comprises cholesterol, at least one phosphatidylethanolamine, at least one milk sphingomyelin, at least one phosphatidylcholine, at least one diacylglycerol, at least one triacylglycerol, at least one phosphatidylinositols, lysobisphosphatidic acid and at least one phosphatidylserine. In a further embodiment the lipid vesicle comprises cholesterol in an amount in the range from 10 to 25 mol %, sphingomyelins in an amount in the range from 4 to 10 mol %, less than 5 mol % of diacylglycerols, less than 5 mol % of triacylglycerols and less than 5 mol % of phosphatidylinositol, and phosphatidylethanolamines in an amount of 10 to 30 mol %, based on the total amount [mol] of the unloaded lipid vesicle as such.

The nucleic acid molecule is a therapeutic or diagnostic nucleic acid molecule. In an embodiment of the present invention, the nucleic acid molecule is a RNAi molecule, such as an siRNA or shRNA. In another embodiment of the present invention, the nucleic acid molecule is a single-stranded antisense molecule, such as modified single-stranded antisense oligonucleotide. The single-stranded antisense oligonucleotide typically has a length of 7 to 30 nucleotides, such as of 10 to 30 nucleotides.

In an embodiment, the nucleic acid molecule, such as the modified single-stranded antisense oligonucleotide has at least one modified internucleoside linkage, such as at least 50% modified internucleoside linkages.

In an embodiment, the modified single-stranded antisense oligonucleotide has at least one modified nucleoside, such as at least 3 2′ sugar modified nucleosides.

The present invention further provides a method for preparing a lipid vesicle carrying a nucleic acid molecule, and a lipid vesicle obtainable or obtained by said method, the composition, wherein the lipid vesicle has a hydrodynamic diameter Dₕ of less than 300 nm, measured by DLS, wherein said lipid vesicle is administered orally, wherein said nucleic acid molecule is for delivery to one or more of the target tissues selected from the group consisting of the central nervous system, spleen, gastrointestinal tract, liver and T-cells.

### Definitions

Lipid Vesicles

Within the meaning of the present invention, the term “lipid vesicle” refers to an artificially, preferably synthetically, prepared vesicle made of at least one lipid bilayer or multilamellar (lipid membrane), wherein the lipid vesicle comprises naturally-derived or synthetic phospholipids or other surfactants or stealth components (reduce macrophage recognition) such as a pegylated lipid (e.g. DMG-PEG2000, DSPE-PEG2000 or other lipid anchor or PEG size), and optionally other membrane components such as cholesterol and proteins. The structure of the lipid vesicle can act as a physical reservoir for pharmaceutically active ingredients such as for the nucleic acid molecule as described herein.

The hydrodynamic diameter (particle size, herein also referred to as “diameter”) of the lipid vesicle of the invention is less than 300 nm, more preferably less than 280 nm, more preferably less than 250 nm, more preferably less than 220 nm, more preferably less than 200 nm, more preferably less than 180 nm, measured by DLS.

Preferably, the hydrodynamic diameter is in the range from 10 to 300 nm, in the range from 20 to 280 nm, more preferably in the range from 30 to 250 nm, more preferably in the range from 40 to 220 nm, more preferably in the range from 50 to 200 nm, more preferably in the range from 60 to 180 nm, more preferably a diameter in the range from 70 to 180 nm measured by DLS.

Preferably, the lipid vesicle as provided herein has a negative to neutral zeta potential under physiological conditions, preferably a zeta potential in the range from −30 to 0 mV, more preferably in the range from −15 to 0, determined with Zetasizer ZSP (Malvern Instruments, Malvern, UK) in 0.1×DPBS at 25° C. measured at a total lipid concentration of 0.1 mM.

In an embodiment, the lipid vesicle is a synthetic lipid vesicle. Accordingly, it has been generated artificially by combining the various components as set forth herein. It is envisaged that it is not isolated from an animal source, e.g. from a mammal. For example, it shall not have been isolated from milk such as bovine milk. The individual components of the lipid vesicle (such as the sphingomyelins) may have been isolated from a natural (e.g. animal or plant) source.

Particle Size/Diameter

The terms “Particle Size” or “hydrodynamic diameter” refer to the averaged/mean hydrodynamic diameter (Dₕ) of the lipid vesicle as measured by Photon Correlation Spectroscopy—herein referred to as Dynamic Light Scattering (DLS). The averaged/mean hydrodynamic diameter (Dₕ) means that in a lipid vesicle composition individual vesicles may fall outside the given range, however the average particle size of the composition is within the give range. The DLS method is based on the scattering of laser light by particles and utilizes the measurement of the speed at which particles are diffusing due to Brownian motion. The particle velocity correlates to the size of particles. In the present application the Photon Correlation Spectroscopy (DLS) was carried out using a Malvern Zetasizer Ultra (Malvern Panalytical, Malvern, UK) at a laser wavelength of 685 nm. Scattered light was detected at an angle of 173°. Results are expressed as mean±SD of n=3 measurements in DPBS (No. 14190250, Thermo Scientific, Waltham, Mass., USA) at room temperature. For lipid vesicle stability assessment, unloaded vesicles have been used.

Stability

Preferably, the lipid vesicle according to the invention is stable in gastric fluid. The term “stable in gastric fluid” as used within the present application is defined as a change (i.e. decrease or increases) in the mean hydrodynamic diameter (Dₕ) of the lipid vesicle by at most 90%, such as at most 86% (when compared to the respective initial Dh measured as described above), when incubated in simulated gastric fluid [SGF] (NaCl 34 mM, HCl 0.83 M, 0.1% Triton X-100 (pH 1.2)) at a lipid vesicle concentration of 1 mM for at least 5 h at a temperature of 20° C. Preferably, the mean hydrodynamic diameter (Dₕ) of the lipid vesicle at time 0 or 5 h after incubation in gastric fluid is less than 300 nm, such as in the range from 50 to 250 nm.

According to an embodiment, the hydrodynamic diameter of the lipid vesicle only changes by at most 60%, such as 50%, such as 40%, such as 30%, more preferably by at most 25%, when incubated in simulated gastric fluid [SGF] (NaCl 34 mM, HCl 0.83 M, 0.1% Triton X-100 (pH 1.2)) at a concentration of 1 mM for at least 5 h at a temperature of 20° C. Preferably, the mean hydrodynamic diameter (Dₕ) of the lipid vesicle at time 0 and after 5 h incubation in gastric fluid is less than 300 nm, such as in the range from 60 to 180 nm. In a preferred embodiment the size changes at most by 25% and includes decreases as well as increases of the size and the mean hydrodynamic diameter (Dₕ) of the lipid vesicle at time 0 or 5 h after the incubation in gastric fluid is in the range from 100 to 165 nm.

Preferably, the mean hydrodynamic diameter (Dₕ) of the lipid vesicle after the incubation in SGF is less than 300 nm, such as in such as in the range from 50 to 250 nm, and preferably in the range from 60 to 180 nm, and more preferably in the range from 100 to 165 nm.

Moreover, the lipid vesicle according to the invention is preferably also stable in fasted state intestinal fluid. The term “stable in fasted state intestinal fluid” as used within the present application is defined as a change (i.e. decrease or increases) in the mean hydrodynamic diameter (Dₕ) in of the lipid vesicle by at most 40% when incubated in simulated fasted state intestinal fluid [FaSSIF] (NaH₂PO₄ 28.6 mM, Taurocholic acid sodium salt 3 mM, Lecithin 0.75 mM, NaCl 105.8 mM (pH 6.5) at a concentration of 1 mM for at least 3 h, such as at least 5 h, at a temperature of 20° C.

Preferably, the hydrodynamic diameter of the lipid vesicles changes by at most 40%, more preferably by at most 35%, more preferably by at most 30%, more preferably by at most 25%, more preferably by at most 200%, more preferably at most 15%, more preferably at most 10%, when incubated in simulated fasted state intestinal fluid [FaSSIF] (NaH₂PO₄ 28.6 mM, Taurocholic acid sodium salt 3 mM, Lecithin 0.75 mM, NaCl 105.8 mM (pH 6.5) at a concentration of 1 mM for at least 3 h, such as at least, 5 h at a temperature of 20° C. Preferably, the mean hydrodynamic diameter (Dₕ) of the lipid vesicle at time 0 and 5 h after incubation in FaSSIF is less than 300 nm, such as in the range from 50 to 275, such as in the range from 60 to 180. In a preferred embodiment the size changes at most by 15% and includes decreases as well as increases of the size and the mean hydrodynamic diameter (Dₕ) of the lipid vesicle at time 0 and 5 h after the incubation in FaSSIF is in the range from 100 to 150 nm. In a preferred embodiment the stable lipid vesicle is within parameters set forth above for both SGF and FaSSIF.

In a further preferred embodiment a stable lipid vesicle is defined fulfil the following parameters: after incubation in SGF the size change is less than 55% and includes decreases as well as increases of the size and the mean hydrodynamic diameter (Dₕ) of the lipid vesicle at time 0 and 5 h after the incubation in SGF is in the range from 80 to 165 nm and after incubation in FaSSIF the size change is less than 25% and includes decreases as well as increases of the size and the mean hydrodynamic diameter (Dₕ) of the lipid vesicle at time 0 and 5 h after the incubation in FaSSIF is in the range from 80 to 150 nm

In a further preferred embodiment a stable lipid vesicle is defined fulfil the following parameters: after incubation in SGF the size change is less than 25% and includes decreases as well as increases of the size and the mean hydrodynamic diameter (Dₕ) of the lipid vesicle at time 0 and 5 h after the incubation in SGF is in the range from 100 to 165 nm and after incubation in FaSSIF the size change is less than 15% and includes decreases as well as increases of the size and the mean hydrodynamic diameter (Dₕ) of the lipid vesicle at time 0 and 5 h after the incubation in FaSSIF is in the range from 100 to 150 nm.

The Components of the Lipid Vesicle

Preferred components of the lipid vesicles (as defined above) are described in more detail here. If not indicated otherwise, the preferred amount of each respective component of the lipid vesicle is given in mol %, based on the total amount of the unloaded lipid vesicle [in mol], i.e. the amount of the vesicle (without the nucleotide).

Cholesterol

The term cholesterol as used within the meaning of the present invention includes esterified as well as non-esterified cholesterol.

For preferred lipid vesicles of the invention cholesterol is present in an amount of 4 to 50 mol %, more preferably in an amount from 5 to 45 mol %, more preferably in an amount from 10 to 25 mol %, more preferably in an amount from 4 to 20 mol %, more preferably in an amount from 14 to 35 mol % based on the total amount of the unloaded lipid vesicle as such in mol.

Phosphatidylethanolamine

The term “phosphatidylethanolamine” refers to a phosphoglyceride having a phosphoryl ethanolamine head group. The term “at least one phosphatidylethanolamine” as used within the meaning of the present invention includes one specific phosphatidylethanolamine as well as mixtures of two or more different phosphatidylethanolamines.

Phosphatidylethanolamines may have the following structure:

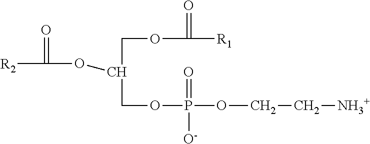

The R₁ and R₂ residues are fatty acid residues, typically residues of naturally occurring fatty acids or of naturally occurring fatty acid derivatives. In another embodiment, the fatty acid residues are residues derived from saturated fatty acid moieties. “Saturated” refers to the absence of a double bond in the hydrocarbon chain. Non-limiting examples of suitable fatty acids are 14:0; 15:0, 16:0, 16:1, 17:0, 18:0, 18:1, 18:2, 18:3, 19:0, 20:0, 20:1, 20:2, 20:3, 10:4, 22:0. 22:1, 22:4, 22:6, 24:0 and 24:1. Thus, R₁ and R₂ are preferably, independently of each other selected from the group consisting of 14:0; 15:0, 16:0, 16:1, 17:0, 18:0, 18:1, 18:2, 18:3, 19:0, 20:0, 20:1, 20:2, 20:3, 20:4, 22:0. 22:1, 22:4, 22:6, 24:0 and 24:1. Preferred combinations of R₁ and R₂ are two fully saturated fatty acids (e.g. 14:0, 14:0, 14:0, 16:0 etc.) in any possible fatty acid length combination, one fully fatty acid plus one unsaturated fatty acid with at least one double bond (e.g. 14:0, 14:1, 14:0, 16:1 etc.) or two unsaturated fatty acid with at least one double bond (e.g. 14:1, 14:1, 14:1, 16:1 etc.).

It is to be understood the that the lipid vesicle may comprise a mixture of two or more different phosphatidylethanolamines.

The phosphatidylethanolamine may be a naturally-occurring phosphatidylethanolamine or a synthetic phosphatidylethanolamine. Preferably, the phosphatidylethanolamine is a naturally-occurring phosphatidylethanolamine.

Non-limiting examples of phosphatidylethanolamines are dimethyl dimyristoyl phosphatidylethanolamine (DMPE), dipalmitoyl-phosphatidylethanolamine (DPPE), distearoyl phosphatidylethanolamine (DSPE), L-alpha-phosphatidyl-ethanolamine, (2-dioleoyl phosphatidylethanolamine (DOPE)), and phosphatidylethanolamines modified with any of the fatty acid moieties enumerated hereinabove. Preferably, the phosphatidylethanolamine is L-alpha-phosphatidyl-ethanolamine.

For most, such as all, lipid vesicles of the invention phosphatidylethanolamine(s) is present in an amount of 2 to 70 mol % lipid vesicle, such as 10 to 70 mol %, such as 10 to 50 mol %, more preferably 10 to 30 mol %, more preferably 19 to 23.5 mol %, based on the total amount of the unloaded lipid vesicle as such in mol.

Phosphatidylethanolamine and Cholesterol

According to an embodiment of the invention, the lipid vesicle according to the invention comprises cholesterol and at least one phosphatidylethanolamine Preferably, the lipid vesicle comprises a total amount of phosphatidyl-ethanolamine and cholesterol in the range from 2 to 90 mol %, such as from 30 to 90 mol %, preferably from 30 to 80 mol %, more preferably from 31 to 65 mol %, based on the total amount of the unloaded lipid vesicle as such in mol and is calculated as the sum of phosphatidylethanolamine(s) and cholesterol. Preferably the phosphatidyl-ethanolamine is L-alpha-phosphatidyl-ethanolamine.

Preferably, the lipid vesicle comprises cholesterol in an amount from 5 to 50 mol %, based on the total amount in mol of the unloaded lipid vesicle as such. According to an embodiment of the invention, the lipid vesicle comprises cholesterol in an amount from 4 to 50 mol %, more preferably in an amount from 5 to 45 mol %, more preferably in an amount from 10 to 25 mol %, more preferably in an amount from 4 to 20 mol %, more preferably in an amount from 14 to 35 mol % and a total sum of phosphatidylethanolamine(s) and cholesterol is in the range from 2 to 90 mol %, such as 30 to 80 mol %, more preferably from 31 to 65 mol %, based on the total amount of each lipid vesicle, and preferably with the phosphatidylethanolamine(s) being L-alpha-phosphatidyl-ethanolamine.

Preferably, the lipid vesicle comprises phosphatidylethanolamine(s), more preferably L-alpha-phosphatidylethanolamine, in an amount of 2 to 70 mol %, based on the total amount of the unloaded lipid vesicle as such. More preferably, the lipid vesicle comprises phosphatidylethanolamine(s), in particular L-alpha-phosphatidylethanolamine, in an amount of 10 to 70 mol %, based on the total amount of the unloaded lipid vesicle as such.

According to one preferred embodiment of the invention, the lipid vesicle comprises phosphatidylethanolamine(s), more preferably L-alpha-phosphatidylethanolamine, in an amount of 10 to 70, more preferably 10 to 50 mol %, more preferably 10 to 30 mol %, more preferably 19 to 23.5 mol % and a total sum of phosphatidylethanolamine(s) and cholesterol from 2 to 90 mol %, such as 30 to 80 mol %, more preferably from 31 to 65 mol %, based on the total amount of the unloaded lipid vesicle as such.

In a further embodiment the lipid vesicle comprises cholesterol in an amount from 4 to 50 mol %, more preferably in an amount from 5 to 45 mol %, more preferably in an amount from 10 to 25 mol %, more preferably in an amount from 4 to 20 mol %, more preferably in an amount from 14 to 35 mol % and phosphatidylethanolamine(s), more preferably L-alpha-phosphatidylethanolamine, in an amount of 2 to 70 mol %, more preferably in an amount from 10 to 70 mol %, more preferably in an amount from 10 to 50 mol %, more preferably 10 to 30 mol %, more preferably 19 to 23.5 mol % with the total sum of phosphatidylethanolamine(s) and cholesterol from 2 to 90 mol %, such as from 30 to 90 mol %, such as from 30 to 80 mol %, more preferably from 31 to 65 mol %, based on the total amount of the unloaded lipid vesicle as such.

In a preferred embodiment the lipid vesicle comprises cholesterol in an amount from 4 to 50 mol % and phosphatidylethanolamine(s) 10 to 70 mol % with the total sum of phosphatidylethanolamine(s) and cholesterol from 30 to 80 mol %, more preferably from 31 to 65 mol % based on the total amount of the unloaded lipid vesicle as such.

Phosphatidylcholine

The term “phosphatidylcholine” refers to a phosphoglyceride having a phosphorylcholine head group. The term “at least one phosphatidylcholine” as used within the meaning of the present invention includes one specific phosphatidylcholine as well as mixtures of two or more different phosphatidylcholines.

Phosphatidylcholine compounds typically have the following structure:

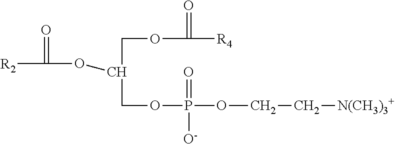

The R₃ and R₄ residues are fatty acid residues, typically residues of naturally occurring fatty acids or of naturally occurring fatty acid derivatives. In another embodiment, the fatty acid residues are residues derived from saturated fatty acid moieties. “Saturated” refers to the absence of a double bond in the hydrocarbon chain. Non-limiting examples of suitable fatty acids are Caprylic acid, CH3(CH2)6COOH,8:0; Capric acid, CH3(CH2)8COOH,10:0; Lauric acid, CH3(CH2)10COOH, 12:0; Myristic acid, CH₃(CH₂)₁₂COOH, 14:0; Palmitic acid, CH₃(CH₂)₁₄COOH, 16:0; Stearic acid, CH₃(CH₂)₁₆COOH, 18:0; Arachidic acid, CH₃(CH₂)₂₂COOH, 20:0; Behenic acid, CH₃(CH₂)₂₀COOH, 22:0; Lignoceric acid, CH₃(CH₂)₂₂COOH, 24:0; Cerotic acid, CH₃(CH₂)₂₄COOH, 26:0, as well as 15:0, 16:1, 17:0, 18:1, 18:2, 18:3, 19:0, 20:1, 20:2, 20:3, 20:4, 22:1, 22:4, 22:6, 24:0 and 24:1.

Thus, R₃ and R₄ are preferably, independently of each other selected from the group consisting of 8:0, 10:0, 12:0, 14:0; 15:0, 16:0, 16:1, 17:0, 18:0, 18:1, 18:2, 18:3, 19:0, 20:0, 20:1, 20:2, 20:3, 20:4, 22:0. 22:1, 22:4, 22:6, 24:0 and 24:1. It is to be understood that the lipid vesicle may comprise mixtures of two or more different phosphatidylcholines.

The at least one phosphatidylcholine may be a naturally-occurring phosphatidylcholine or a synthetic phosphatidylcholine. Preferably, the phosphatidylcholine is a symmetric phosphatidylcholine (i.e. a phosphatidylcholine wherein the two fatty acid moieties are identical).

Non-limiting examples of phosphatidylcholines are soybean phosphatidylcholine, egg phosphatidylcholine (Egg-PC), two (behenic acid) phosphatidylcholine (DBPC), 1,2-distearoyl-sn-glycero-3-phosphocholine (DSPC), Dipalmitoyl-phosphatidylcholine (DPPC), Dimyristoyl-phosphatidylcholine (DMPC), 1,2-dilauroyl-sn-glycero-3-phosphocholine (DLPC), dioleoyl-phosphatidylcholine (DOPC), 1-palmitoyl-2-oleoyl-phosphatidylcholine (POPC), and phosphatidylcholines modified with any of the fatty acid moieties enumerated hereinabove.

As used herein the term “soy phosphatidylcholine” refers to a plurality of unsaturated and saturated fatty acid composition of phosphatidylcholine. In an embodiment, the soy phosphatidylcholine has been isolated from soybean plants, e.g. from soybean seeds. Typically, the soy phosphatidylcholine comprises a mixture of phophatidylcholines comprising fatty acid residues selected from the group consisting of 16:0, 18:0, 18:1, 18:2 and 18:3. In an embodiment, the soy phosphatidylcholine comprises a mixture with phophatidylcholines comprising fatty acid residues derived from 16:0, 18:0, 18:1, 18:2 and 18:3.

In a further embodiment, the, soy phosphatidylcholine comprises about 12%-about 33 mol % palmitic acid (16:0); from about 3%-about 12% mol % stearic acid (C18:0); about 4%-to about 40 mol % of oleic acid (18:1); from about 17%-about 66 mol % of linoleic acid (18:2); about 2%-to about 10 mol % of linolenic acid (18:3) (Szuhaj, B. F. (Ed.). (1989). Lecithins: Sources, Manufacture and Uses (AOCS Monograph), American Oil Chemists' Society, ISBN 9780935315271, Urbana, USA.).

As used in this application, the term “egg phosphatidylcholine” refers to composition of L-alpha-phosphatidyl choline, including, but not limited to, various saturated and unsaturated fatty acids. Preferably, egg phosphatidylcholine comprises about 33 mol % of palmitic acid; about 10% mol % of stearic acid; about 31% mol % of oleic acid; about 18 mol % present in an amount of linoleic acid.

In an embodiment, the at least one phosphatidylcholine is Egg-PC and/or DLPC and/or DOPC, in particular Egg-PC and/or DOPC

Further, it is envisaged that the phosphatidylcholine is not DSPC or DLPC, thus the lipid vesicle preferably does not comprise DSPC and/or does not comprise DLPC, preferably neither DSCP nor DLCP.

Further it is envisaged that the phosphatidylcholine is synthetically derived and comprises both mixed saturated and unsaturated fatty acid groups (e.g. 14:0-16:0; 14:0-18:0; 16:0-14:0; 16:0-18:0; 16:0-18:1; 16:0-18:2; 16:0-20:4; 16:0-22:6; 18:0-14:0; 18:0-16:0; 18:0-18:1; 18:0-18:2; 18:0-20:4; 18:0-22:6; 18:1-14:0; 18:1-16:0; 18:1-16:0; 18:1-18:0; 16:0-20:0).

For lipid vesicles of the invention phosphatidylcholine is present in an amount of 0 to 40 mol % lipid vesicle, such as 1.9 to 40 mol %, such as 4 to 36 mol %, preferably 4 to 15 mol % preferably 20 to 36 mol %, based on the total amount of the unloaded lipid vesicle as such in mol.

Phosphatidylserine

The term “phosphatidylserine” refers to a phospholipid comprising two fatty acids attached in ester linkage to the first and second carbon of glycerol and serine attached through a phosphodiester linkage to the third carbon of the glycerol. The term “at least one phosphatidylserine” as used within the meaning of the present invention includes one specific phosphatidylserine as well as mixtures of two or more different phosphatidylserines.

Phosphatidylserines may have the following structure:

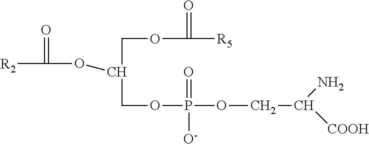

The R₅ and R₆ residues are the fatty acid residues attached via an ester linkage, typically residues of naturally occurring fatty acids or of naturally occurring fatty acid derivatives. In another embodiment, the fatty acid residues are residues derived from saturated fatty acid moieties. “Saturated” refers to the absence of a double bond in the hydrocarbon chain. Non-limiting examples of suitable fatty acids are 8:0, 10:0, 12:0, 14:0; 15:0, 16:0, 16:1, 17:0, 18:0, 18:1, 18:2, 18:3, 19:0, 20:0, 20:1, 20:2, 20:3, 20:4, 22:0. 22:1, 22:4, 22:6, 24:0 and 24:1, in particular 16:0, 18:0, 18:1, 18:2, 18:3, 20:4 and 22:6. Thus, R₅ and R₆ are preferably, independently of each other selected from the group consisting of 8:0, 10:0, 12:0, 14:0; 15:0, 16:0, 16:1, 17:0, 18:0, 18:1, 18:2, 18:3, 19:0, 20:0, 20:1, 20:2, 20:3, 20:4, 22:0. 22:1, 22:4, 22:6, 24:0 and 24:1. It is to be understood the lipid vesicle may comprise a mixtures of two or more different phosphatidylserines.

The at least one phosphatidylserine may be a naturally-occurring or a synthetic phosphatidylserine. Preferably, the phosphatidylserine is a symmetric phosphatidylserine (i.e. a phosphatidylserine wherein the two fatty acid moieties are identical).

Non-limiting examples of phosphatidylserine are L-alpha brain PS (typically comprising fatty acid residues derived from 18:0, 18:1 and 20:4, 22:6), or soy PS (typically comprising fatty acid residues derived from 16:0, 18:0, 18:1, 18:2 and 18:3). Preferably, the phosphatidylserine is 1,2-Diacyl-sn-glycero-3-phospho-L-serine (L-alpha-PS), preferably from soy beans. Preferably, the phosphatidylserine is 1,2-Diacyl-sn-glycero-3-phospho-L-serine (L-alpha-PS), preferably from bovine brain (typically comprising fatty acid residues derived from 18:0, 18:1, 20:4, and 22:6).

For lipid vesicles of the invention phosphatidylserine is present in an amount of 0 to 45 mol % lipid vesicle, such as 2 to 20 mol %, such as 4 to 36 mol %, preferably 12 to 20 mol % preferably 20 to 31 mol %, based on the total amount of the unloaded lipid vesicle as such in mol.

Lysobisphosphatidic Acid

The term “lysobisphosphatidic acid” also known as Bis(monoacylglycero)phosphate (‘BMP’) refers to a negatively charged phospholipid, more specifically a glycerol-phospholipid. BMP was first isolated from rabbit lung but is now known to be a common, but minor constituent of all animal tissues. Its stereochemical configuration differs from that of other animal glycero-phospholipids in that the phosphodiester moiety is linked to positions sn-1 and sn-1′ of glycerol, rather than to position sn-3. It remains unclear whether positions sn-3 and 3′ or sn-2 and sn-2′ in the glycerol moieties are esterified with fatty acids.

Non-limiting examples of lysobisphosphatidic acid are 18:1 BMP (R,R), sn-(1-oleoyl-2-hydroxy)-glycerol-3-phospho-sn-3′-(1′-oleoyl-2′-hydroxy)-glycerol; 18:1 BMP (S,S), sn-(3-oleoyl-2-hydroxy)-glycerol-1-phospho-sn-1′-(3′-oleoyl-2′-hydroxy)-glycerol; 14:0 BMP (S,R), bis(monomyristoylglycero)phosphate (S,R Isomer); 18:1 BMP (S,R), bis(monooleoylglycero)phosphate (S,R Isomer).

Preferably, the lysobisphosphatidic acid is bis(monooleoylglycero)phosphate (S,R Isomer).

Preferably, the lipid vesicle comprises the at least one lysobisphosphatidic acid in an amount in the range from 0 to 12 mol %, such as from 4 to 12 mol %, more preferably in an amount from 4 to 6 mol %, more preferably in an amount from 10 to 12 mol % based on the total amount of the lipid vesicle (in [mol]).

Phosphatidylcholine, Phosphatidylserine and Lysobisphosphatidic Acid

The lipid vesicle described above and below preferably further comprises at least one phosphatidylcholine and/or at least one phosphatidylserine and/or at least one lysobisphosphatidic acid.

Preferably, the sum of phosphatidylcholine and phosphatidylserine and lysobisphosphatidic acid present in the lipid vesicle is in the range from 10 to 54 mol % calculated as sum of phosphatidylcholine(s) and phosphatidylserine(s) and Lysobisphosphatidic acid(s) and based on the total amount of the lipid vesicle (in [mol]). Preferably, the lipid vesicle comprises the at least one phosphatidylcholine in an amount in the range from 0 to 45 mol %, e.g. in an amount from 1.9 to 40 mol %, such as 10 to 40 mol %, such as 4 to 36 mol %, preferably 4 to 15 mol % preferably 20 to 36 mol % and based on the total amount of the lipid vesicle (in [mol]). Preferably, the lipid vesicle comprises the at least one phosphatidylserine in an amount in the range of 0 to 45 mol % lipid vesicle, such as 2 to 20 mol %, such as 4 to 36 mol %, preferably 12 to 20 mol %, preferably from 14 to 42 mol %, more preferably in an amount from 14 to 20 mol %, preferably 20 to 31 mol %, based on the total amount of the unloaded lipid vesicle as such in mol.

Preferably, the lipid vesicle comprises the at least one lysobisphosphatidic acid in an amount in the range from 0 to 12 mol %, such as from 4 to 12 mol %, more preferably in an amount from 4 to 6 mol %, more preferably in an amount from 10 to 12 mol % based on the total amount of the lipid vesicle (in [mol]).

Preferably, the lipid vesicle comprises >0 mol %, such as from 2 to 40 mol %, phosphatidylcholine, wherein more preferably the phosphatidylcholine is Egg-PC and/or DOPC. If the lipid vesicle comprises at least one phosphatidylcholine, the phosphatidylcholine is preferably comprised in an amount from 4 to 36 mol %, based on the total amount of the unloaded lipid vesicle and calculated as sum of all phosphatidycholines present in the lipid vesicle.

In a further embodiment the lipid vesicle comprises
i) cholesterol in an amount from 4 to 50 mol %, more preferably in an amount from 5 to 45 mol %, more preferably in an amount from 10 to 25 mol %, more preferably in an amount from 4 to 20 mol %, more preferably in an amount from 14 to 35 mol % and
ii) phosphatidylethanolamine(s), more preferably L-alpha-phosphatidylethanolamine, in an amount of 2 to 70 mol %, more preferably in an amount from 10 to 70 mol %, more preferably in an amount from 10 to 50 mol %, more preferably 10 to 30 mol %, more preferably 19 to 23.5 mol %
with the total sum of phosphatidylethanolamine(s) and cholesterol is from 30 to 80 mol % based on the total amount of the unloaded lipid vesicle as such and
iii) phosphatidylcholine and phosphatidylserine and/or lysobisphosphatidic acid present in the range from 10 to 54 mol % calculated as sum of phosphatidylcholine(s) and phosphatidylserine(s) and Lysobisphosphatidic acid(s) and based on the total amount of the lipid vesicle (in [mol]).

In a preferred embodiment the lipid vesicle comprises
i) cholesterol in an amount from 4 to 50 mol % and
ii) phosphatidylethanolamine(s) 10 to 70 mol %
with the total sum of phosphatidylethanolamine(s) and cholesterol from 30 to 80 mol %, more preferably from 31 to 65 mol % based on the total amount of the unloaded lipid vesicle as such; and
iii) phosphatidylcholine in an amount in the range from 0 to 40 mol %, such as from 4 to 36 mol %.
iv) phosphatidylserine in an amount in the range from 0 to 45 mol % lipid vesicle, such as from 12 to 20 mol %;
v) lysobisphosphatidic acid in an amount in the range from 0 to 12 mol %, such as from 4 to 12
with the total sum of phosphatidylcholine and phosphatidylserine and/or lysobisphosphatidic acid present in the range from 10 to 54 mol % based on the total amount of the unloaded lipid vesicle as such.

Further Components

As outlined above, the lipid vesicle may further comprise at least one sphingomyelin and/or diacylglycerols and/or triacylglycerols and/or phosphatidylinositols.

Sphingomyelin

The term “sphingomyelin” is known to the skilled person and refers to lipids usually comprising phosphocholine head group, a sphingosine and a fatty acid. It is one of the few membrane phospholipids not synthesized from glycerol.

The sphingomyelin may be selected from the group consisting of milk sphingomyelin, brain sphingomyelin, egg sphingomyelin, synthetic sphingomyelins and mixtures of two or more thereof, or fully synthetically derived sphingomyelin. The sphingomyelin may e.g. comprise fatty acids derivatives derived from fatty acids such as 8:0, 10:0, 12:0, 14:0; 15:0, 16:0, 16:1, 17:0, 18:0, 18:1, 18:2, 18:3, 19:0, 20:0, 20:1, 20:2, 20:3, 20:4, 22:0. 22:1, 22:4, 22:6, 23:0, 24:0 and 24:1.

For example, the sphingomyelin is a brain sphingomyelin, such as a porcine or monkey brain sphingomyelin, preferably typically comprising a mixture of sphingomyelins comprising fatty acids derivatives derived from fatty acids such as 16:0, 18:0, 20:0, 22:0, 24:0 and 24:1.

Or, the sphingomyelin is an egg sphingomyelin, preferably typically comprising a mixture of sphingomyelins comprising fatty acids derivatives derived from fatty acids such as 16:0, 18:0 and 24:0.

Or, the sphingomyelin is a milk sphingomyelin, preferably typically comprising a mixture of sphingomyelins comprising fatty acids derivatives derived from fatty acids such as 16:0, 22:0, 23:0, 24:0, 24:1.

Preferably, the sphingomyelin may be a milk sphingomyelin.

If the lipid vesicle comprises at least one sphingomyelin, the lipid vesicle comprises at least one sphingomyelin preferably in an amount in the range from 2 to 45 mol %, such as from 15 to 45, such as from 4 to 25 mol %, more preferably in amount from 2 to 15 mol %, more preferably in an amount from 4 to 13 mol %, more preferably in an amount from 2 to 7 mol % based on the total amount of unloaded the lipid vesicle (in mol) as such.

In a further embodiment the lipid vesicle comprises
i) cholesterol in an amount from 4 to 50 mol %, more preferably in an amount from 5 to 45 mol %, more preferably in an amount from 10 to 25 mol %, more preferably in an amount from 4 to 20 mol %, more preferably in an amount from 14 to 35 mol %, and
ii) phosphatidylethanolamine(s), more preferably L-alpha-phosphatidylethanolamine, in an amount of 2 to 70 mol %, more preferably in an amount from 10 to 70 mol %, more preferably in an amount from 10 to 50 mol %, more preferably 10 to 30 mol %, more preferably 19 to 23.5 mol % with the total sum of phosphatidylethanolamine(s) and cholesterol is from 30 to 80 mol % based on the total amount of the unloaded lipid vesicle as such; and
iii) at least one sphingomyelin, in an amount in the range from 2 to 45 mol %, such as from 15 to 45, such as from 4 to 25 mol %, more preferably in amount from 2 to 15 mol %, more preferably in an amount from 4 to 13 mol %.

In a further embodiment the lipid vesicle comprises
i) cholesterol in an amount from 4 to 50 mol % and
ii) phosphatidylethanolamine(s) 10 to 70 mol %
with the total sum of phosphatidylethanolamine(s) and cholesterol from 30 to 80 mol %, more preferably from 31 to 65 mol % based on the total amount of the unloaded lipid vesicle as such; and
iii) phosphatidylcholine and phosphatidylserine and/or lysobisphosphatidic acid present in the range from 10 to 54 mol % calculated as sum of phosphatidylcholine(s) and phosphatidylserine(s) and Lysobisphosphatidic acid(s) and based on the total amount of the lipid vesicle (in [mol]); and
iv) at least one sphingomyelin, in an amount in the range from 2 to 45 mol %, such as from 15 to 45, such as from 4 to 25 mol %, more preferably in amount from 2 to 15 mol %, more preferably in an amount from 4 to 13 mol %.

In a preferred embodiment the lipid vesicle comprises
i) cholesterol in an amount from 4 to 50 mol % and
ii) phosphatidylethanolamine(s) 10 to 70 mol %
with the total sum of phosphatidylethanolamine(s) and cholesterol from 30 to 80 mol %, more preferably from 31 to 65 mol % based on the total amount of the unloaded lipid vesicle as such; and
iii) at least one sphingomyelin, in an amount in the range from 2 to 40 mol %, such as from 4 to 13.

In a preferred embodiment the lipid vesicle comprises
i) cholesterol in an amount from 4 to 50 mol % and
ii) phosphatidylethanolamine(s) 10 to 70 mol %
with the total sum of phosphatidylethanolamine(s) and cholesterol from 30 to 80 mol %, more preferably from 31 to 65 mol % based on the total amount of the unloaded lipid vesicle as such; and
iii) phosphatidylcholine in an amount in the range from 0 to 40 mol %, such as from 4 to 36 mol %,
iv) phosphatidylserine in an amount in the range from 0 to 45 mol %, such as from 12 to 20 mol %,
v) lysobisphosphatidic acid in an amount in the range from 0 to 12 mol % lipid vesicle, such as 4 to 12 mol %
with the total sum of phosphatidylcholine and phosphatidylserine and/or lysobisphosphatidic acid present in the range from 10 to 54 mol % based on the total amount of the unloaded lipid vesicle as such; and
vi) at least one sphingomyelin, in an amount in the range from 2 to 45 mol %, such as from 2 to 15 mol %, more preferably in an amount from 4 to 13 mol %.

Diacylglycerol

The term “diacylglycerol” (DAG) refers to a glyceride comprising two fatty acid residues covalently linked to glycerol through ester linkages. Two possible forms exist, 1,2-diacylglycerols and 1,3-diacylglycerols. Although 1,2-diacyl-rac-glycerols are useful, the diacylglycerols are preferably 1,2-diacylglycerols, and most preferably 1,2-diacyl-sn-glycols.

Representative saturated free fatty acids (fatty acids) from which the fatty acid residue attached to the glycerol may be derived from include, but are not limited to: methanoic (formic); ethanoic (acetic); propanoic (propionic); butanoic (butyric); pentanoic (valeric); hexanoic (caproic); heptanoic (enanthic); octanoic (caprylic); nonanoic (pelargonic); decanoic (capric); undecanoic (undecylic); dodecanoic (lauric); tridecanoic (tridecylic); tetradecanoic (myristic); pentadecanoic (pentadecylic); hexadecanoic (palmitic); heptadecanoic (margaric); octadecanoic (stearic); nonadecanoic (nonadecylic); eicosanoic (arachidic); heneicosanoic; docosanoic (behenic); tricosanoic; tetracosanoic; pentacosanoic; hexacosanoic (cerotic); heptacosanoic; octacosanoic (montanic); nonacosanoic; triacontanoic (melissic) acid; and the like.

Preferably, the diacylglycerol, if present, is dipalmitin, the fatty acid residues attached to the glycerol are thus preferably derived from hexadecanoic acid (Palmitinic acid).

Preferably, the lipid vesicle comprises the at least one diacylglycerol in an amount in the range from 0 to 5 mol %, such as from 3 to 5 mol % based on the total amount of the lipid vesicle (in [mol]).

Triacylglycerol

The term “triacylglycerol” (TAG) refers to a glyceride comprising three fatty acid residues covalently linked to glycerol through ester linkages.

Representative saturated free fatty acids (fatty acids) from which the fatty acid residue attached to the glycerol may be derived from include, but are not limited to: methanoic (formic), ethanoic (acetic), propanoic (propionic), butanoic (butyric), pentanoic (valeric), hexanoic (caproic), heptanoic (enanthic), octanoic (caprylic), nonanoic (pelargonic), decanoic (capric), undecanoic (undecylic), dodecanoic (lauric), tridecanoic (tridecylic), tetradecanoic (myristic), pentadecanoic (pentadecylic), hexadecanoic (palmitic), heptadecanoic (margaric), octadecanoic (stearic), nonadecanoic (nonadecylic), eicosanoic (arachidic), heneicosanoic, docosanoic (behenic), tricosanoic, tetracosanoic, pentacosanoic, hexacosanoic (cerotic), heptacosanoic, octacosanoic (montanic), nonacosanoic, triacontanoic (melissic) acid, and the like.

Preferably, the triglycerol (TAG) is Glycerol tristearate, the fatty acid residues attached to the glycerol are thus preferably derived from octadecanoic acid.

Preferably, the lipid vesicle comprises the at least one triglycerol in an amount in the range from 0 to 5 mol %, such as from 3 to 5 mol % based on the total amount of the lipid vesicle (in [mol]).

Phosphatidylinositol

The term “phosphatidylinositol” or “Pl” refers to an acidic phospholipid that contains an inositol attached to the phosphate group of phosphatidic acid.

The phosphatidylinositol may be a natural phosphatidylinositol or a synthetic phosphatidylinositol. As the natural phosphatidylinositol e.g. L-a-phosphatidylinositol (sodium salt) (e.g., from bovine liver or soy), L-a-phosphatidylinositol-4-phosphate (ammonium salt) (e.g., from porcine brain) (Brain PI(4)P), or L-a-phosphatidylinositol-4,5-bisphosphate (ammonium salt) (e.g. from porcine brain) (Brain PI(4,5)P2) may be mentioned. Preferably, the phosphatidylinositols comprise fatty acids selected from the group consisting of 16:0, 18:0, 18:1, 18:2, 18:3, 20:3, 20:4 and combinations thereof.

Preferably, the phosphatidylinositol is L-a-phosphatidylinositol, more preferably L-a-phosphatidylinositol (bovine liver (“Liver Pl”).

Preferably, the lipid vesicle comprises the at least one triglycerol in an amount in the range from 0 to 5 mol %, such as from 3 to 5 mol % based on the total amount of the lipid vesicle (in [mol]).

Preferably, in case the lipid vesicle comprises at least one diacylglycerol and/or at least one triacylglycerol and/or at least one phosphatidylinositol wherein the total amount of the sum of diacylglycerols and triacylglycerols and phosphatidylinositols in an amount in the range from 0 to 15 mol % of the lipid vesicle.

Preferably, the lipid vesicle comprises an amount in the range from 0 to 5 mol % of diacylglycerols and 0 to 5 mol % of triacylglycerols and 0 to 5 mol % phosphatidylinositols.

In a further embodiment the lipid vesicle comprises
i) cholesterol in an amount from 4 to 50 mol %, more preferably in an amount from 5 to 45 mol %, more preferably in an amount from 10 to 25 mol %, more preferably in an amount from 4 to 20 mol %, more preferably in an amount from 14 to 35 mol %, and
ii) phosphatidylethanolamine(s), more preferably L-alpha-phosphatidylethanolamine, in an amount of 2 to 70 mol %, more preferably in an amount from 10 to 70 mol %, more preferably in an amount from 10 to 50 mol %, more preferably from 10 to 30 mol %, more preferably from 19 to 23.5 mol %
with the total sum of phosphatidylethanolamine(s) and cholesterol is from 30 to 80 mol % based on the total amount of the unloaded lipid vesicle as such; and
iii) at least one diacylglycerol(s) in an amount in the range from 0 to 5 mol %;
iv) at least one of triacylglycerol(s) in an amount in the range from 0 to 5 mol %; and
v) at least one phosphatidylinositol(s) in an amount in the range from 0 to 5 mol %.

In a further embodiment the lipid vesicle comprises
i) cholesterol in an amount from 4 to 50 mol %, more preferably in an amount from 5 to 45 mol %, more preferably in an amount from 10 to 25 mol %, more preferably in an amount from 4 to 20 mol %, more preferably in an amount from 14 to 35 mol %, and
ii) phosphatidylethanolamine(s), more preferably L-alpha-phosphatidylethanolamine, in an amount of 2 to 70 mol %, more preferably in an amount from 10 to 70 mol %, more preferably in an amount from 10 to 50 mol %, more preferably 10 to 30 mol %, more preferably 19 to 23.5 mol %
with the total sum of phosphatidylethanolamine(s) and cholesterol is from 30 to 80 mol % based on the total amount of the unloaded lipid vesicle as such; and
iii) phosphatidylcholine and phosphatidylserine and/or lysobisphosphatidic acid present in the range from 10 to 54 mol % calculated as sum of phosphatidylcholine(s) and phosphatidylserine(s) and Lysobisphosphatidic acid(s) and based on the total amount of the lipid vesicle (in [mol]); and
iv) at least one sphingomyelin, in an amount in the range from 2 to 45 mol %, such as from 15 to 45, such as from 4 to 25 mol %, more preferably in amount from 2 to 15 mol %, more preferably in an amount from 4 to 13 mol %; and
v) at least one diacylglycerol and/or at least one triacylglycerol and/or at least one phosphatidylinositol wherein the total amount of the sum of diacylglycerols and triacylglycerols and phosphatidylinositols in an amount in the range from 0 to 15 mol % of the lipid vesicle.

In a preferred embodiment the lipid vesicle comprises
i) cholesterol in an amount from 4 to 50 mol % and
ii) phosphatidylethanolamine(s) 10 to 70 mol %
with the total sum of phosphatidylethanolamine(s) and cholesterol from 30 to 80 mol %, more preferably from 31 to 65 mol % based on the total amount of the unloaded lipid vesicle as such; and
iii) at least one diacylglycerol(s) in an amount in the range from 0 to to 5 mol %;
iv) at least one of triacylglycerol(s) in an amount in the range from 0 to 5 mol %; and
v) at least one phosphatidylinositol(s) in an amount in the range from 0 to 5 mol %.

In a preferred embodiment the lipid vesicle comprises
i) cholesterol in an amount from 4 to 50 mol % and
ii) phosphatidylethanolamine(s) 10 to 70 mol %
with the total sum of phosphatidylethanolamine(s) and cholesterol from 30 to 80 mol %, more preferably from 31 to 65 mol % based on the total amount of the unloaded lipid vesicle as such; and
iii) phosphatidylcholine in an amount in the range from 0 to 40 mol %, such as from 4 to 36 mol %,
iv) phosphatidylserine in an amount in the range from 0 to 45 mol %, such as from 12 to 20 mol %,
v) lysobisphosphatidic acid in an amount in the range from 0 to 12 mol % lipid vesicle, such as 4 to 12 mol %
with the total sum of phosphatidylcholine and phosphatidylserine and/or lysobisphosphatidic acid present in the range from 10 to 54 mol % based on the total amount of the unloaded lipid vesicle as such; and
vi) at least one sphingomyelin, in an amount in the range from 2 to 45 mol %, such as from 2 to 15 mol %, more preferably in an amount from 4 to 13 mol %.
vii) at least one diacylglycerol and/or at least one triacylglycerol and/or at least one phosphatidylinositol wherein the total amount of the sum of diacylglycerols and triacylglycerols and phosphatidylinositols in an amount in the range from 0 to 15 mol % of the lipid vesicle.

Nucleic Acid Molecule

The lipid vesicle provided herein shall carry a nucleic acid molecule such as an oligonucleotide (e.g. an antisense oligonucleotide) or an RNA interference (RNAi) molecule.

The term “nucleic acid molecule” or “therapeutic nucleic acid molecule” as used herein is defined as it is generally understood by the skilled person, as a molecule comprising two or more covalently linked nucleosides (i.e. a nucleotide sequence). The nucleic acid molecule(s) referred to in the method of the invention are generally therapeutic oligonucleotides below 50 nucleotides in length. The nucleic acid molecules may be or comprise an antisense oligonucleotide, or may be another nucleic acid molecule, such as a CRISPR RNA, a siRNA, shRNA, an aptamer, or a ribozyme. Therapeutic nucleic acid molecules are commonly made in the laboratory by solid-phase chemical synthesis followed by purification and isolation. shRNA's are however often delivered to cells using lentiviral vectors (see for example Soan and Yang 2010 N Am J Med Sci 2(12): 598) which are then transcribed to produce the single stranded RNA that will form a stem loop (hairpin) RNA structure that is capable of interacting with the RNA interference machinery (including the RNA-induced silencing complex (RISC)). When referring to a sequence of the nucleic acid molecule, reference is made to the sequence or order of nucleobase moieties, or modifications thereof, of the covalently linked nucleotides or nucleosides. The nucleic acid molecule of the invention is man-made, and is chemically synthesized, and is typically purified or isolated. In some embodiments the nucleic acid molecule of the invention is not a shRNA transcribed from a vector upon entry into the target cell. The nucleic acid molecule of the invention may comprise one or more modified nucleosides or nucleotides.

In some embodiments, the nucleic acid molecule of the invention comprises or consists of 7 to 60 nucleotides in length, such as from 8 to 60, such as from 10 to 55, such as from 12 to 50 such as from 13 to 45, such as from 14 to 40, such as from 15 to 30, such as from 16 to 22, such as from 16 to 18 or 15 to 17 contiguous nucleotides in length.

In some embodiments, the nucleic acid molecule or contiguous nucleotide sequence thereof comprises or consists of 24 or less nucleotides, such as 22, such as 20 or less nucleotides, such as 18 or less nucleotides, such as 14, 15, 16 or 17 nucleotides. It is to be understood that any range given herein includes the range endpoints. Accordingly, if a nucleic acid molecule is said to include from 12 to 30 nucleotides, both 12 and 30 nucleotides are included.

In some embodiments, the contiguous nucleotide sequence comprises or consists of 12, 13, 14, 15, 16, 17, 18, 19, 20, 21 or 22 contiguous nucleotides in length

The nucleic acid molecule(s) are for modulating the expression of a target nucleic acid in a mammal. In some embodiments the nucleic acid molecules, such as for siRNAs, shRNAs and antisense oligonucleotides, are typically for inhibiting the expression of a target nucleic acid(s).

In one embodiment of the invention the nucleic acid molecule is selected from a RNAi agent, such as a siRNA or shRNA. In another embodiment the nucleic acid molecule is a single stranded antisense oligonucleotide, such as a high affinity modified antisense oligonucleotide interacting with RNaseH.

In some embodiments the nucleic acid molecule comprises phosphorothioate internucleoside linkages.

In some embodiments the nucleic acid molecule may be conjugated to non-nucleosidic moieties (conjugate moieties).

A library of nucleic acid molecules is to be understood as a collection of variant nucleic acid molecules. The purpose of the library of nucleic acid molecules can vary. In some embodiments, the library of nucleic acid molecules is composed of oligonucleotides with overlapping nucleobase sequence targeting one or more mammalian FUBP1 target nucleic acids with the purpose of identifying the most potent sequence within the library of nucleic acid molecules. In some embodiments, the library of nucleic acid molecules is a library of nucleic acid molecule design variants (child nucleic acid molecules) of a parent or ancestral nucleic acid molecule, wherein the nucleic acid molecule design variants retaining the core nucleobase sequence of the parent nucleic acid molecule.

Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by oligonucleotide synthesis followed by purification and isolation. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides, such as 2′ sugar modified nucleosides.

Antisense Oligonucleotides

The term “Antisense oligonucleotide” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self complementarity is less than 50% across of the full length of the oligonucleotide

Advantageously, the single stranded antisense oligonucleotide of the invention does not contain RNA nucleosides, since this will decrease nuclease resistance.

Advantageously, the antisense oligonucleotide of the invention comprises one or more modified nucleosides or nucleotides, such as 2′ sugar modified nucleosides. Furthermore, it is advantageous that the nucleosides which are not modified are DNA nucleosides.

RNAi Molecule

Herein, the term “RNA interference (RNAi) molecule” refers to short double-stranded RNA molecule capable of inducing RNA-dependent gene silencing via the RNA-induced silencing complex (RISC) in a cell's cytoplasm, where they interact with the catalytic RISC component argonaute. One type of RNAi molecule is a small interfering RNA (siRNA), which is a double-stranded RNA molecule composed of two complementary oligonucleotides, where the binding of one strand to complementary mRNA after transcription, leads to its degradation and loss of translation. A small hairpin RNA (shRNA) is a single stranded RNA molecule that forms a stem loop (hairpin) structure which upon expression is able to reduce mRNA via the DICER and RNA reducing silencing complex (RISC). RNAi molecules can be designed based on the sequence of the gene of interest (target nucleic acid). Corresponding RNAi can then be synthesized chemically or by in vitro transcription, or expressed from a vector or PCR product.

shRNA molecules are generally between 40 and 70 nucleotides in length, such as between 45 and 65 nucleotides in length, such as 50 and 60 nucleotides in length, and interacts with the endonuclease known as Dicer which is believed to processes dsRNA into 19-23 base pair short interfering RNAs with characteristic two base 3′ overhangs which are then incorporated into an RNA-induced silencing complex (RISC). siRNA molecules are double stranded, with each strand being between 18 and 35 nucleotides in length, such as 20 to 30 nucleotides in length, such as 22 to 27 nucleotides in length. siRNA's are often designed with a two base 3′ overhang to resemble the product produced by Dicer, which forms the RISC substrate. Effective extended forms of Dicer substrates have been described in U.S. Pat. Nos. 8,349,809 and 8,513,207, hereby incorporated by reference. Upon binding to the appropriate target mRNA, one or more endonucleases within the RISC cleave the target to induce silencing. RNAi oligonucleotides may be chemically modified using modified internucleotide linkages and 2′ sugar modified nucleosides, such as 2′-4′ bicyclic ribose modified nucleosides, including LNA and cET or 2′ substituted modifications like of 2′-O-alkyl-RNA, 2′-O-methyl-RNA, 2′-alkoxy-RNA, 2′-O-methoxyethyl-RNA (MOE), 2′-amino-DNA, 2′-fluoro-DNA, arabino nucleic acid (ANA), 2′-fluoro-ANA and tricyclo DNA (TcDNA; Nature Medicine 2015, 3(21), 270-275).

In some embodiments RNAi nucleic acid molecules comprise one or more phosphorothioate internucleoside linkages. In RNAi molecules phosphorothioate internucleoside linkages may reduce or the nuclease cleavage in RICS it is therefore advantageous that not al internucleoside linkages are modified. Phosphorothioate internucleoside linkages can advantageously be place in the 3′ and/or 5′ end of the RNAi nucleic acid molecule, in particular in the of the part of the molecule that is not complementary to the target nucleic acid (e.g. the sense stand or passenger strand in an siRNA molecule). The region of the RNAi molecule that is complementary to the target nucleic acid (e.g. the antisense or guide strand in an siRNA molecule) may however also be modified in the first 2 to 3 internucleoside linkages in the 3′ and/or 5′ terminal.

Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the oligonucleotide comprises the contiguous nucleotide sequence which hybridized to the target nucleic acid, such as a F-G-F′ gapmer region, and may optionally comprise further nucleotide(s), for example a nucleotide linker region which may be used to attach a functional group to the contiguous nucleotide sequence. The nucleotide linker region may or may not be complementary to the target nucleic acid. Adventurously, the contiguous nucleotide sequence is 100% complementary to the target nucleic acid.

Nucleotides

Nucleotides are the building blocks of oligonucleotides and polynucleotides, and for the purposes include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA containing oligonucleotides, 5-methyl cytosine LNA nucleosides may be used.

Modified Internucleoside Linkages

The term “modified internucleoside linkage” is defined as generally understood by the skilled person as linkages other than phosphodiester (PO) linkages, that covalently couples two nucleosides together. The oligonucleotides of the invention may therefore comprise modified internucleoside linkages. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region of a gapmer oligonucleotide, as well as in regions of modified nucleosides, such as region F and F′.

In an embodiment, the oligonucleotide comprises one or more internucleoside linkages modified from the natural phosphodiester, such one or more modified internucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages.

In an embodiment, the modified single stranded antisense oligonucleotide comprises at least one modified internucleoside linkage. In some embodiments at least 50% of the internucleoside linkages in the oligonucleotide as referred to herein, or contiguous nucleotide sequence thereof, are modified internucleoside linkages, such as at least 60%, such as at least 70%, such as at least 80% or such as at least 90% of the internucleoside linkages in the oligonucleotide, or contiguous nucleotide sequence thereof, are modified internucleoside linkages. In some embodiments all of the internucleoside linkages of the oligonucleotide, or contiguous nucleotide sequence thereof, are modified internucleoside linkages.

In some embodiments all of the internucleoside linkages of the oligonucleotide, or contiguous nucleotide sequence thereof, are nuclease resistant internucleoside linkages. It will be recognized that, in some embodiments the nucleosides which link the oligonucleotide of the invention to a non-nucleotide functional group, such as a conjugate, may be phosphodiester.

Preferably, the modified internucleoside linkage is a phosphorothioate internucleoside linkage, a diphosphorothioate internucleoside linkage, or a boranophosphate internucleoside linkage.

A preferred modified internucleoside linkage is a phosphorothioate internucleoside linkage. Phosphorothioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorothioate linkages, such as at least 60%, such as at least 70%, such as at least 80% or such as at least 90% of the internucleoside linkages in the oligonucleotide, or contiguous nucleotide sequence thereof, are phosphorothioate linkages. In some embodiments all of the internucleoside linkages of the oligonucleotide, or contiguous nucleotide sequence thereof, are phosphorothioate linkages.

Nuclease resistant linkages, such as phosphorothioate linkages, are particularly useful in oligonucleotide regions capable of recruiting nuclease when forming a duplex with the target nucleic acid, such as region G for gapmers. Phosphorothioate linkages may, however, also be useful in non-nuclease recruiting regions and/or affinity enhancing regions such as regions F and F′ for gapmers, or in mixmers and totalmers. Gapmer oligonucleotides may, in some embodiments comprise one or more phosphodiester linkages in region F or F′, or both region F and F′, which the internucleoside linkage in region G may be fully phosphorothioate.

Advantageously, all the internucleoside linkages in the contiguous nucleotide sequence of the oligonucleotide are phosphorothioate linkages.

It is recognized that, as disclosed in EP2 742 135, antisense oligonucleotide may comprise other internucleoside linkages (other than phosphodiester and phosphorothioate), for example alkyl phosphonate/methyl phosphonate internucleoside, which according to EP2 742 135 may for example be tolerated in an otherwise DNA phosphorothioate gap region.

Modified Nucleoside

The term “modified nucleoside” or “nucleoside modification” as used herein refers to nucleosides modified as compared to the equivalent DNA or RNA nucleoside by the introduction of one or more chemical modifications of the moieties forming the backbone of the oligonucleotide, such as modifications to the sugar moiety or substitution of the sugar moiety with alternative chemical structures. The term modified nucleoside may also be used herein interchangeably with the term “nucleoside analogue” or modified “units” or modified “monomers”.

Numerous nucleosides with modification of the ribose sugar moiety have been made, primarily with the aim of improving certain properties of nucleic acid molecules, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798).

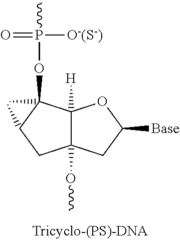

Modified nucleosides also include nucleosides where the sugar moiety is replaced with a non-sugar moiety, for example in the case of peptide nucleic acids (PNA), or morpholino nucleic acids.

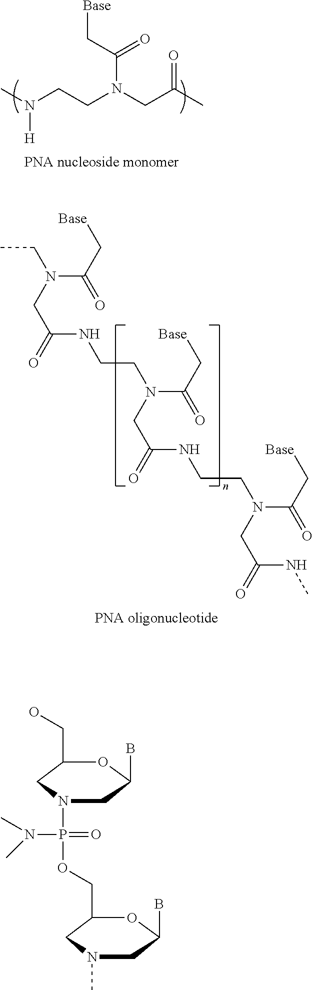

Phosphorodiamidate Morpholino Oligomer (PMO)

Sugar modifications also include modifications made via altering the substituent groups on the ribose ring to groups other than hydrogen, or the —OH groups naturally found in RNA or DNA nucleosides. Substituents may, for example be introduced at the 2′, 3′, 4′ or 5′ positions Nucleosides with an unmodified DNA or RNA sugar moiety are termed DNA or RNA nucleosides herein.

Nucleosides with modifications in the base region of the DNA or RNA nucleoside are still generally termed DNA or RNA if they allow Watson Crick base pairing.

2′ Sugar Modified Nucleosides.

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

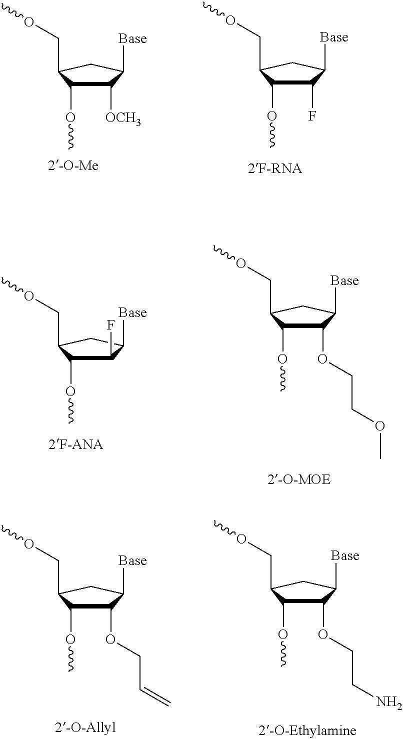

In addition to the ribose based nucleoside modifications, the so called tricycle DNA (TcDNA) is a further addition to the above list of nucleoside modifications

In relation to the present invention 2′ substituted does not include 2′ bridged molecules like LNA.

Modified Antisense Oligonucleotide

As used in herein a modified antisense oligonucleotide is an oligonucleotide where the oligonucleotide backbone has been modified compared to the natural occurring RNA or DNA oligonucleotides. The oligonucleotide backbone refers to the internucleotide linkage and/or the sugar moiety, but generally not to modifications in the base moieties.

Modification of the antisense oligonucleotide backbone is commonly used for therapeutic oligonucleotides to increase stability, in particular towards nucleases or to increase affinity towards the target nucleic acid.

Stability increasing modifications are for example modified internucleoside linkages, some sugar modifications as well as peptide backbones. Modifications increasing affinity are for example sugar modifications.

The modified single stranded antisense oligonucleotide as referred to herein comprises one or more modified nucleosides and/or modified internucleoside linkages. Modified oligonucleotides can comprise a mixture of 2′ sugar modified nucleotides and DNA or RNA nucleosides, for example in a gapmer or mixmer design these are also sometimes termed “chimeric” oligonucleotides. PNA and morpholino oligonucleotides are generally composed of the same backbone, such that they are composed of PNA moieties or morpholino moieties only.

The modified oligonucleotide may in some embodiments comprise or consist of the contiguous nucleotide sequence of the oligonucleotide which is complementary to the target nucleic acid, such as the gapmer, mixmer or totalmer region, and further 5′ and/or 3′ nucleosides which may not be fully complementary to the target nucleic acid. Such additional terminal nucleosides can for example be 2 to 4 phosphodiester linked DNA nucleosides and serve as a biocleavable linker between the contiguous nucleotide sequence complementary to the target nucleic acid and for example a conjugate moiety.

The modified single-stranded antisense oligonucleotide typically has a length of 7 to 35 nucleotides. In an embodiment, the modified single-stranded antisense oligonucleotide has a length of 7 to 30 nucleotides. In another embodiment, the modified single-stranded antisense oligonucleotide has a length of 10 to 30 nucleotides. In another embodiment, the modified single-stranded antisense oligonucleotide has a length of 14 to 30 nucleotides. In another embodiment, the modified single-stranded antisense oligonucleotide has a length of 14 to 20 nucleotides. In another embodiment, the modified single-stranded antisense oligonucleotide has a length of 7 to 14 nucleotides. In another embodiment, the modified single-stranded antisense oligonucleotide has a length of 8 to 12 nucleotides.

The term antisense oligonucleotide in the singular form “an antisense oligonucleotide” or “a modified antisense oligonucleotide” refers to a population of oligonucleotides which share a common nucleobase sequence and pattern of chemical modifications and are typically derived from a common manufacturing process.

Locked Nucleic Acids (LNA)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Further non limiting, exemplary LNA nucleosides are disclosed in Scheme 1.

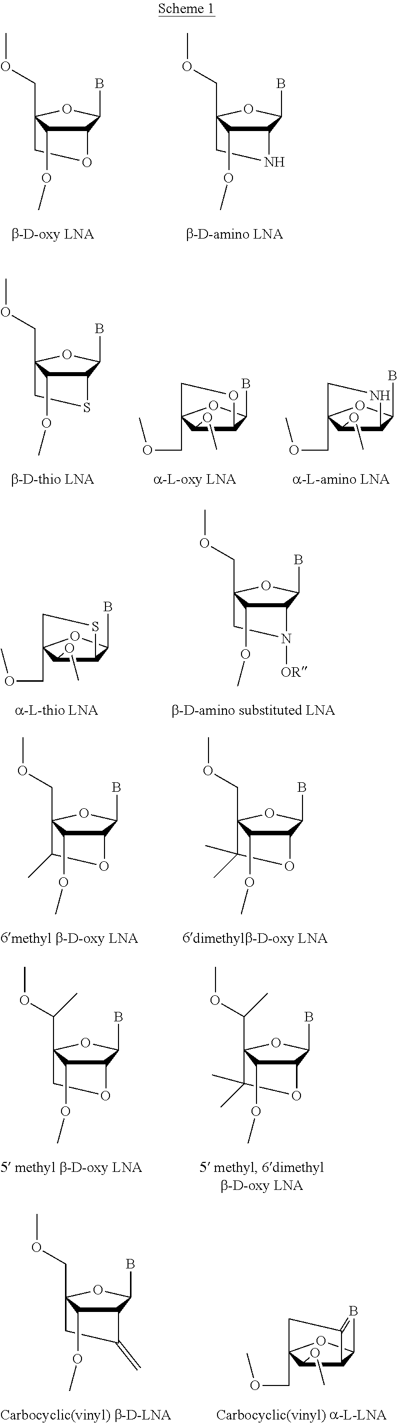

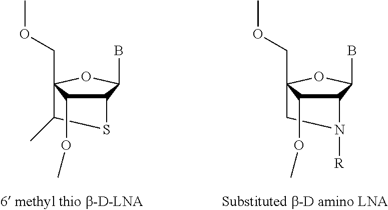

Particular LNA nucleosides are beta-D-oxy-LNA, 6′-methyl-beta-D-oxy LNA such as (S)-6′-methyl-beta-D-oxy-LNA (ScET) and ENA.

A particularly advantageous LNA is beta-D-oxy-LNA.

Gapmer

The modified single stranded antisense oligonucleotide comprised by the lipid vesicle provided herein may be a gapmer, also termed gapmer oligonucleotide or gapmer designs.

The antisense gapmers are commonly used to inhibit a target nucleic acid via RNase H mediated degradation. A gapmer oligonucleotide comprises at least three distinct structural regions a 5′-flank, a gap and a 3′-flank, F-G-F′ in the ’5->3′ orientation. The “gap” region (G) comprises a stretch of contiguous DNA nucleotides which enable the oligonucleotide to recruit RNase H, such as from 5-16 contiguous DNA nucleotides. The gap region is flanked by a 5′ flanking region (F) comprising one or more sugar modified nucleosides, advantageously high affinity 2′ sugar modified nucleosides, and by a 3′ flanking region (F′) comprising one or more sugar modified nucleosides, advantageously high affinity 2′ sugar modified nucleosides. The flanking regions independently consist of 1-8 contiguous nucleotides with a sugar modified nucleoside defining each end. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, such as 70%, 75%, 80%, 85%, 90% or 100%. A DNA is always positioned adjacent to a sugar modified nucleoside of the 5′ (F) or 3′ (F′) region respectively. The Gap region may in some instances contain modified nucleosides which do not prevent RNase H cleavage, for example, alpha-L-LNA, C4′ alkylated DNA (as described in WO/2009/090182 and Vester et al., Bioorg. Med. Chem. Lett. 18 (2008) 2296-2300, both incorporated herein by reference), arabinose derived nucleosides like ANA and 2F-ANA (Mangos et al. 2003 J. AM. CHEM. SOC. 125, 654-661), UNA (unlocked nucleic acid) (as described in Fluiter et al., Mol. Biosyst., 2009, 10, 1039 incorporated herein by reference. The flanks may further be defined by having at least one sugar modified nucleoside at the end most distant from the gap region, i.e. at the 5′ end of the 5′ flank and at the 3′ end of the 3′ flank.

Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 32 nucleosides, such as 13 to 24, such as 14 to 22 nucleosides, Such as from 14 to 20, such as 16 to 18 nucleosides.

By way of example, the gapmer oligonucleotide of the present invention can be represented by the following formulae:

F₁₋₈-G₅₋₁₆-F′₁₋₈, such as

F₁₋₈-G₇₋₁₆-F′₂₋₈

with the proviso that the overall length of the gapmer regions F-G-F′ is at least 12, such as at least 14 nucleotides in length.

LNA Gapmer

The modified single stranded antisense oligonucleotide by the lipid vesicle provided herein may be a LNA gapmer. An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In a classic LNA gapmer both flanks consist of LNA nucleosides. However alternating flank LNA gapmers have also been described, where one or both of the flanking regions comprise both LNA and DNA nucleosides (see for example WO2016/127002). In such alternating designs, the flanking region F or F′, or both F and F′ comprise at least three nucleosides, wherein the 5′ and 3′ most nucleosides of the F and/or F′ region are LNA nucleosides.

In some embodiments the LNA gapmer is of formula: [LNA]₁₋₅-[G]₆₋₁₆-[LNA]₁₋₅, wherein G is the RNase recruiting gap region. LNA gapmers with a 3-10-3 (LNA-DNA-LNA) design has been widely used in the prior art.

MOE Gapmers

The modified single stranded antisense oligonucleotide by the lipid vesicle provided herein may be a MOE gapmer. A MOE gapmers is a gapmer wherein regions F and F′ consist of MOE nucleosides. In some embodiments the MOE gapmer is of design [MOE]₁₋₈-[G]₅₋₁₆-[MOE]₁₋₈, such as [MOE]₂₋₇-[G]₆₋₁₄-[MOE]₂₋₇, such as [MOE]₃₋₆-[G]₈₋₁₂-[MOE]₃₋₆, wherein G is the RNase recruiting gap region. MOE gapmers with a 5-10-5 design (MOE-DNA-MOE) have been widely used in the art.

Mixed Wing Gapmer

The modified single stranded antisense oligonucleotide comprised by the lipid vesicle provided herein may be a mixed wing gapmer. A mixed wing gapmer is an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units. In some embodiments at least one of region F and F′, or both region F and F′ comprise at least one LNA nucleoside, the remaining nucleosides of region F and F′ are independently selected from the group consisting of MOE and LNA. In some mixed wing embodiments, one or both of region F and F′ may further comprise one or more DNA nucleosides.

Mixed wing gapmer designs are disclosed in WO2008/049085 and WO2012/109395, both of which are hereby incorporated by reference.

Totalmer

The modified single stranded antisense oligonucleotide comprised by the lipid vesicle provided herein may be a totalmer. A totalmer is a single stranded modified antisense oligonucleotide, or contiguous nucleotide sequence thereof, which does not comprise DNA or RNA nucleosides, and generally only comprises one type of nucleoside analogues. The contiguous nucleotide sequence of a totalmer oligonucleotide is generally between 7 and 16 nucleotides, such as between 8 and 12 2′ sugar modified nucleosides which can be selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA (locked nucleic acid) nucleosides.

Generally short totalmers (below 10 nucleotides in length) are composed of 100% LNA units. For longer totalmers one or more of the nucleoside units may be selected from the non-LNA nucleotide analogues such as 2′ sugar substituted nucleosides referred to herein.

In some embodiments the totalmer consist or comprises of a contiguous nucleotide sequence which consists only of LNA units.

PNA and morpholino oligonucleotides can also be considered as totalmers.

Totalmer designs have shown to be effective as therapeutic oligonucleotides, particularly when targeting microRNA (antimiRs) or as splice switching oligonucleotides (SSOs).

Mixmer

The modified single stranded antisense oligonucleotide comprised by the lipid vesicle provided herein may be a mixmer. A mixmer is a single stranded modified antisense oligonucleotide, or contiguous nucleotide sequences thereof, where as opposed to gapmers, there is no contiguous sequence of more than 5 naturally occurring DNA nucleosides, and therefore it does not recruit RNase H.

Generally, the mixmer comprises or consists of a contiguous nucleotide sequence of repeating pattern of one type of nucleoside analogue, such as a 2′ sugar modified nucleoside, and a naturally occurring nucleoside, such as DNA. This could for example be a repeating pattern of LNA nucleosides and DNA nucleosides starting and ending with an LNA in the 5′ end and 3′ end. The mixmer may however also combine different types of nucleoside analogues, such that the repeating pattern for instance is every second or every third nucleoside is a nucleoside analogue, such as LNA, and the remaining nucleosides are naturally occurring nucleosides, such as DNA, or a 2′ sugar substituted nucleoside analogue such as 2′MOE of 2′fluoro analogues or other 2′ sugar substituted nucleosides mentioned herein. The mixmer generally has a nucleoside analogue (independently selected) at the 5′ or 3′ termini. Examples of mixmers are described in WO2007/027894, WO2007/112754, WO2007/112754 (antimiRs) and WO2008/131807 (SSOs). The distribution pattern of nucleoside analogues and DNA is subject to optimization depending on the oligonucleotide sequence.

Mixmer designs are highly effective as therapeutic oligonucleotides, particularly when targeting microRNA (antimiRs), microRNA binding sites on mRNAs (Blockmirs) or as splice-switching oligomers (SSOs).

AntimiR

The modified single stranded antisense oligonucleotide comprised by the lipid vesicle provided herein may be an antimiR. An antimiR, also known as an anti-miRNA oligonucleotide, is an antisense oligonucleotide capable of controlling the in vivo activity of a microRNA (miRNA). miRNAs are complementary sequences (approximately 22 bp) to mRNA and are involved in the cleavage of RNA or the suppression of the translation. The antimiRs primarily function by sequestering the mature miRNA in competition with the cellular target mRNAs thereby leading to functional inhibition of the miRNA and de-repression of the direct targets (see for example WO2009/043353 and Stenvang et al 2012 Silence 3:1). AntimiRs are designed with an increased binding affinity for the microRNA and increased nuclease resistance to improve their ability of prevent binding of the microRNA to the target mRNA. AntimiRs are generally designed as totalmers or mixmers not inducing RNase H cleavage.

Blockmir

The modified single stranded antisense oligonucleotide comprised by the lipid vesicle provided herein may be a blockmir. A blockmir, also known as an antagomir, is an antisense oligonucleotide that hat prevent other molecules from binding to a desired site on an mRNA molecule. This can for example be the microRNA binding sites on a mRNA thereby preventing its regulation by the miRNA's. So where an antimiR targets the miRNA directly the blockmir only targets a specific mRNA preventings it interaction with for example miRNAs. Blockmirs are generally designed as totalmers or mixmers not inducing RNase H cleavage.

Splice-Switching Antisense Oligonucleotide (SSO)

The modified single stranded antisense oligonucleotide comprised by the lipid vesicle provided herein may be a splice-switching oligonucleotide. A splice-switching oligonucleotide (SSOs) is an antisense oligonucleotide that base-pair with a pre-mRNA and disrupt the normal splicing repertoire of the transcript by blocking the RNA-RNA base-pairing or protein-RNA binding interactions that occur between components of the splicing machinery and the pre-mRNA. Modulation of splicing is particularly valuable in cases of disease caused by mutations that lead to disruption of normal splicing or when interfering with the normal splicing process of a gene transcript may be therapeutic (see for example Havens and Hastings 2016 Nucleic Acid Research 44:6549). Currently there are two approved SSO molecules on the market, Exondys 51 (morpholino SSO) to treat Duchenne muscular dystrophy and Spinraza (MOE totalmer SSO) to treat spinal muscular atrophy. SSOs are generally designed as totalmers or mixmers not inducing RNase H cleavage.

Conjugate

The term conjugate as used herein refers to an oligonucleotide which is covalently linked to a non-nucleotide moiety (conjugate moiety or region C or third region).

Conjugation of the single stranded antisense oligonucleotide to a lipid may improve the encapsulation of the single stranded antisense oligonucleotide into the lipid vesicle by increasing the hydrophobicity of the oligonucleotide.

In one embodiment the single stranded antisense oligonucleotide which is to be encapsulated into a lipid vesicle is conjugated at the 5′ or 3′ terminal with a lipophilic conjugate moiety. The lipohphilic conjugate moiety may be selected from the group consisting of sterols, stanols, steroids, polycyclic aromatic groups, aliphatic groups, lipids, phospholipids, lipophilic alcohols, fatty acids and fatty esters.

In some embodiments, the conjugate moiety is or comprises a lipid, a phospholipid e.g. di-hexadecyl-rac-glycerol ortriethylammonium 1,2-di-o-hexadecyl-rac-glycero-3-h-phosphonate or a lipophilic alcohol, a palmityl moiety, a cationic lipid, a neutral lipid, sphingolipids, and fatty acids such as stearic, oleic, elaidic, linoleic, linoleaidic, linolenic, and myristic acids. In some embodiments the fatty acid comprises a C4-C30 saturated or unsaturated alkyl chain. The alkyl chain may be linear or branched.

Lipophilic conjugate moieties include, for example, sterols stanols, and steroids and related compounds such as cholesterol (U.S. Pat. No. 4,958,013 and Letsinger et al., Proc. Natl. Acad. Sci. USA, 1989, 86, 6553), thiocholesterol (Oberhauser et al, Nucl Acids Res., 1992, 20, 533), Ianosterol, coprostanol, stigmasterol, ergosterol, calciferol, cholic acid, deoxycholic acid, estrone, estradiol, estratriol, progesterone, stilbestrol, testosterone, tocopherol, androsterone, deoxycorticosterone, cortisone, 17-hydroxycorticosterone, their derivatives, and the like. In some embodiments, the conjugate may be selected from the group consisting of cholesterol, thiocholesterol, hexylamino-carbonyl-oxycholesterol, lanosterol, coprostanol, stigmasterol, ergosterol, calciferol, cholic acid, deoxycholic acid, estrone, estradiol, estratriol, progesterone, stilbestrol, testosterone, androsterone, deoxycorticosterone, cortisone, and 17-hydroxycorticosterone. In some embodiments the conjugate moiety comprises tocopherol. In some embodiments, the conjugate moiety comprises cholesterol.

Other lipophilic conjugate moieties include aliphatic groups, such as, for example, straight chain, branched, and cyclic alkyls, alkenyls, and alkynyls. The aliphatic groups can have, for example, 5 to about 50, 6 to about 50, 8 to about 50, or 10 to about 50 carbon atoms. Example aliphatic groups include e.g., dodecandiol or undecyl residues, dodecyl, hexadecyl, heptadecyl, octadecyl, nonadecyl, terpenes, bornyl, adamantyl, derivatives thereof and the like. In some embodiments, one or more carbon atoms in the aliphatic group can be replaced by a heteroatom such as O, S, or N (e.g., geranyloxyhexyl). Further suitable lipophilic conjugate moieties include aliphatic derivatives of glycerols such as alkylglycerols, bis(alkyl)glycerols, tris(alkyl)glycerols, monoglycerides, diglycerides, and triglycerides. In some embodiments, the lipophilic conjugate is di-hexyldecyl-rac-glycerol or 1,2-di-O-hexyldecyl-rac-glycerol (Manoharan et al., Tetrahedron Lett., 1995, 36, 3651; Shea, et al., Nuc. Acids Res., 1990, 18, 3777) or phosphonates thereof. Saturated and unsaturated fatty functionalities, such as, for example, fatty acids, fatty alcohols, fatty esters, and fatty amines, can also serve as lipophilic conjugate moieties. In some embodiments, the fatty functionalities can contain from about 6 carbons to about 30 or about 8 to about 22 carbons. Example fatty acids include, capric, caprylic, lauric, palmitic, myristic, stearic, oleic, linoleic, linolenic, arachidonic, eicosenoic acids and the like.

In further embodiments, lipophilic conjugate moieties can be polycyclic aromatic groups having from 6 to about 50, 10 to about 50, or 14 to about 40 carbon atoms. Example polycyclic aromatic groups include pyrenes, purines, acridines, xanthenes, fluorenes, phenanthrenes, anthracenes, quinolines, isoquinolines, naphthalenes, derivatives thereof and the like. Other suitable lipophilic conjugate moieties include menthols, trityls (e.g., dimethoxytrityl (DMT)), phenoxazines, lipoic acid, phospholipids, ethers, thioethers (e.g., hexyl-S-tritylthiol), derivatives thereof and the like. Preparation of lipophilic conjugates of oligonucleotides are well-described in the art, such as in, for example, Saison-Behmoaras et al, EMBO J., 1991, 10, 1111; Kabanov et al., FEBS Lett., 1990, 259, 327; Svinarchuk et al, Biochimie, 1993, 75, 49; (Mishra et al., Biochim. Biophys. Acta, 1995, 1264, 229, and Manoharan et al., Tetrahedron Lett., 1995, 36, 3651.

Biocleavable Nucleotide Linkers

The single-stranded antisense oligonucleotide which is to be encapsulated into a lipid vesicle may in some embodiments comprise or consist of the contiguous nucleotide sequence of the oligonucleotide which is complementary to the target nucleic acid, such as the gapmer F-G-F′, the totalmer or the mixmer regions and may further comprise additional 5′ and/or 3′ nucleosides. The further 5′ and/or 3′ nucleosides may or may not be fully complementary to the target nucleic acid, such further 5′ and/or 3′ nucleosides may be referred to as region D′ and D″ herein.

The addition of region D′ or D″ may be used for the purpose of joining the contiguous nucleotide sequence, such as the contiguous nucleotide sequence of the gapmer, totalmer or mixmer, to a conjugate moiety or another functional group in a manner that allows removal of the conjugate from the contiguous nucleoside sequence once it has reached its target tissue. To form a biocleavable linker between the contiguous nucleotide sequence and the conjugate moiety the additional nucleosides (region D′ or D″) are linked with a nuclease liable linkage such as a phosphodiester internucleoside linkages. Region D′ or D″ may independently comprise or consist of 1, 2, 3, 4 or 5 additional nucleosides, which may be complementary or non-complementary to the target nucleic acid. The transition between region D′ or D″ and the gapmer, mixmer or totalmer is recognized by the transition from a phosphorothioate linked sugar modified nucleoside to a phosphodiester linked non-sugar modified nucleoside, such as a DNA or RNA or base modified versions of these. In some embodiments the additional 5′ and/or 3′ end nucleotides are linked with phosphodiester linkages, both between the region D′ or D″ nucleosides (DNA or RNA), but also the internucleoside linkage connecting the sugar modified nucleoside at the terminal end of the gapmer, mixmer or totalmer with the additional 5′ and/or 3′ end nucleotides (Region D) is preferably a phosphodiester linkage. In some embodiments the additional 5′ and/or 3′ end nucleotides are phosphodiester linked DNA or RNA, preferably there is at least two, such as at least three, such as at least 5 consecutive phosphodiester linkages between the 5′ end or the 3′ end of a gapmer, mixmer or totalmer and the conjugate moiety.

Nucleotide based biocleavable linkers suitable for use as region D′ or D″ are disclosed in WO2014/076195, which include by way of example a phosphodiester linked DNA dinucleotide.

For the gapmer the designs using a biocleavable nucleotide linker can be described by the following formulas D′-F-G-F′, F-G-F′-D″ or D′-F-G-F′-D″. In this instance the F-G-F′ is the gapmer portion of the oligonucleotide and region D′ or D″ constitute a separate part of the oligonucleotide.

In one embodiment the single stranded antisense oligonucleotide to be encapsulated into the lipid vesicle of the invention comprises a region D′ and/or D″ in addition to the contiguous nucleotide sequence which constitutes the gapmer, mixmer or totalmer.

In some embodiments, the oligonucleotide of the present invention can be represented by the following formulae:

F-G-F′; in particular F₁₋₈-G₅₋₁₆-F′₂₋₈

D′-F-G-F′, in particular D′₁₋₃-F₁₋₈-G₅₋₁₆-F′₂₋₈

F-G-F′-D″, in particular F₁₋₆-G₅₋₁₆-F′₂₋₈-D″₁₋₃

D′-F-G-F′-D″, in particular D′₁₋₃-F₁₋₈-G₅₋₁₆-F′₂₋₆-D″₁₋₃

In some embodiments the internucleoside linkage positioned between region D′ and region F is a phosphodiester linkage. In some embodiments the internucleoside linkage positioned between region F′ and region D″ is a phosphodiester linkage.

Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the number of nucleotides in percent of a contiguous nucleotide sequence in a nucleic acid molecule (e.g. oligonucleotide) which, at a given position, are complementary to (i.e. form Watson Crick base pairs with) a contiguous sequence of nucleotides, at a given position of a separate nucleic acid molecule (e.g. the target nucleic acid or target sequence). The percentage is calculated by counting the number of aligned bases that form pairs between the two sequences (when aligned with the target sequence 5′-3′ and the oligonucleotide sequence from 3′-5′), dividing by the total number of nucleotides in the oligonucleotide and multiplying by 100. In such a comparison a nucleobase/nucleotide which does not align (form a base pair) is termed a mismatch. Preferably, insertions and deletions are not allowed in the calculation of % complementarity of a contiguous nucleotide sequence.

The term “fully complementary”, refers to 100% complementarity.

Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned bases that are identical (a match) between two sequences (e.g. in the contiguous nucleotide sequence of the compound of the invention and in the reference sequence), dividing that number by the total number of nucleotides in the aligned region and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

Target Nucleic Acid

The term “target nucleic acid” as used herein refers to a gene, a RNA, a microRNA, a mRNA, and pre-mRNA, a mature mRNA or a cDNA sequence which is intentionally modulated by a modified single-stranded antisense oligonucleotide as referred to herein. The target nucleic acid of the present invention is at least expressed in the central nervous system (e.g. brain tissue), in the spleen, in the gastrointestinal tract, in the liver and/or in T-cells. It is to be understood that the target nucleic acid is associated with the disease to be treated. Accordingly, the disease to be treated by the isolated lipid vesicle as referred to herein is predicted to be improved by changing the levels of the target nucleic acid or targets directly regulated by the target nucleic acid or the protein encoded by the target nucleic acid. In one embodiment the disease is caused by abnormal levels of the target nucleic acid or a protein encoded by the target nucleic acid. In another embodiment the disease is caused by malfunctioning variants of the protein encoded by the target nucleic acid such as splice variants or mutational variants. In another embodiment the disease may be improved by increasing expression of/from the target nucleic acid compared to normal levels if such an increase, for example, serves to reduce abnormal levels of another disease causing protein. In particular, the disease can be improved by modulating the target nucleic acid, in the central nervous system (e.g. brain tissue), in the spleen, in the gastrointestinal tract, in the liver and/or in T-cells. The expression “abnormal level” is well understood by the skilled person and generally refers to a level of the target nucleic acid or a protein encoded from it which is increased or decreased in a subject suffering from the disease to be treated as compared to a subject not suffering from the disease (in particular, in the central nervous system (e.g. brain tissue), in the spleen, in the gastrointestinal tract, in the liver and/or in T-cells). In some embodiments the disease is treated by down-regulating expression of the target nucleic acid.

The target nucleic acid can be any nucleic acid which is expressed in the cells or tissues referred to above. In an embodiment, the target nucleic acid is a mRNA. In another embodiment, the target nucleic acid is a pre-mRNA. In another embodiment, the target nucleic acid is a long non-coding RNA (lncRNA). In another embodiment, the target nucleic acid is a miRNA.

The contiguous sequence of nucleobases of the modified single-stranded antisense oligonucleotide is typically complementary, such as 80%, such as 90%, such as 95%, such as fully complementary, to the target nucleic acid, as measured across the length of the oligonucleotide. It is advantageous it the modified single-stranded antisense oligonucleotide is fully complementary to the target nucleic acid. Optionally of one or two mismatches to the target nucleic acid can be allowed. The sequence complementarity is generally measured across the full length of the oligonucleotide or preferably across the contiguous nucleotide sequence of the oligonucleotide optionally excluding nucleotide based linker regions which may link the oligonucleotide to an optional functional group such as a conjugate, or other non-complementary terminal nucleotides.

Target Sequence

The term “target sequence” as used herein refers to a sequence of nucleotides present in the target nucleic acid which comprises the nucleobase sequence which is complementary to the oligonucleotide loaded into the lipid vesicle. In some embodiments, the target sequence consists of a region on the target nucleic acid which is complementary to the contiguous nucleotide sequence of the oligonucleotide of the invention.

Modulation of a Target

The term “modulation” as used herein is to be understood as an overall term for an oligonucleotide's ability to alter the amount of target nucleic acid when compared to the amount of target nucleic acid prior to administration of the lipid vesicle of the present invention. Alternatively, modulation of expression may be determined by reference to a control experiment. E.g., the control is an individual or target cell treated with a saline composition or unloaded isolated lipid vesicle.

In one embodiment the modulation is achieved by hybridization of the modified single-stranded antisense oligonucleotide or the RNAi oligonucleotide to the target nucleic acid, thereby either i) down-regulating, i.e. reducing, the expression of said target nucleic acid, ii) effecting splice switching of the target nucleic acid, resulting in expression of a splice variant of the target nucleic acid or iii) blocking the target nucleic acid, e.g. a miRNA or mRNA, to increase the protein expression from a downstream mRNA or the target mRNA.

In another embodiment the modified single-stranded antisense oligonucleotide is an aptamer which binds to a target nucleic acid or target protein via non-covalent interactions such as electrostatic interactions, hydrophobic interactions and their complementary shapes, instead of via Watson-Crick base pairing, and thereby blocks or activates it target.

The modulation of the target is at least 20% compared to the normal expression level of the target, more preferably at least 30%, at least 40%, at least 50%, at least 60%, at least 70%, at least 80%, or at least 90% compared to the normal expression level of the target.

In some embodiments the lipid vesicle loaded oligonucleotide of the invention may be capable of inhibiting expression levels of the target mRNA by at least 40% in vivo following oral administration of lipid vesicles with at least one 1 μM nucleic acid molecule (e.g. the oligonucleotide or the RNAi molecule), such as 5 μM oligonucleotide or RNAi molecule.

In some embodiments the lipid vesicle loaded oligonucleotide may be capable of increasing expression levels of target protein by at least 20% in vivo following oral administration of lipid vesicles with at least one 1 μM nucleic acid molecule (e.g. the oligonucleotide or the RNAi molecule), such as 5 μM oligonucleotide or RNAi molecule.

In some embodiments the lipid vesicle loaded oligonucleotide of the invention may be capable of changing the splice switching of a target nucleic acid to provide an alternatively spliced target protein which constitutes at least 20% of the total target protein in vivo following oral administration of lipid vesicles with at least 1 μM nucleic acid molecule (e.g. the oligonucleotide or the RNAi molecule), such as 5 μM oligonucleotide or RNAi molecule.

Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic. The subject to be treated is preferably a mammal, e.g. a mouse. In an embodiment, the subject is a human subject. Diseases to be treated include central nervous system diseases (such as brain diseases), spleen diseases, gastrointestinal tract diseases, liver diseases and diseases involving the T-cells. Preferred diseases are disclosed elsewhere herein.

### DETAILED DESCRIPTION OF THE INVENTION

In a first aspect, the present invention concerns a lipid vesicle, such as a synthetically prepared lipid vesicle, of less than 300 nm carrying a nucleic acid molecule such as an antisense oligonucleotide or RNAi molecule for use as a medicament, wherein said lipid vesicle is administered orally, i.e. the vesicle is formulated for oral administration.

The term lipid vesicle has been defined in the definitions above. Preferably, said lipid vesicle has a hydrodynamic diameter Dh of less than 300 nm, measured according to DLS, as set out above. Preferably the lipid vesicle has a diameter in the range of 50 to 300 nm. In further embodiments the hydrodynamic diameter Dₕ of the lipid vesicle is less than 200 nm, measured according to DLS, preferably a diameter in the range from 50 to 275 nm, such as from 50 to 250, and preferably from 60 to 180 nm.

According to a first embodiment, the lipid vesicle comprises one or more, such as at least two, at least three or at least four, lipids independently selected from the group consisting of cholesterol, at least one sphingomyelin, at least one phosphatidylcholine, at least one phosphatidylserine, at least one phosphatidylethanolamine, at least one diacylglycerol, at least one triacylglycerol, at least one phosphatidylinositol, at least one lysobisphosphatidic acid and phosphatidylserine.

In further embodiments the lipid vesicle preferably comprises one or more lipids independently selected from the group consisting of:
cholesterol in an amount in the range from 4 to 50 mol %, more preferably in an amount from 5 to 45 mol %, more preferably in an amount from 10 to 25 mol %, more preferably in an amount from 4 to 20 mol %, more preferably in an amount from 14 to 35 mol %,
optionally at least one sphingomyelin in an amount in the range from 0 to 45 mol %, such as from 2 to 45 mol %, such as from 15 to 45 mol %, such as from 4 to 25 mol %, more preferably in amount from 2 to 15 mol %, more preferably in an amount from 4 to 13 mol %,
at least one phosphatidylcholine in an amount in the range from 0 to 40 mol % lipid vesicle, such as 1.9 to 40 mol %, such as 4 to 36 mol %, preferably 4 to 15 mol % preferably 20 to 36 mol %,
at least one phosphatidylethanolamines in an amount in the range from 2 to 70 mol %, more preferably in an amount from 10 to 70 mol %, more preferably in an amount from 10 to 50 mol %, more preferably 10 to 30 mol %, more preferably 19 to 23.5 mol %,
at least one phosphatidylserine in an amount in the range from 0 to 45 mol % lipid vesicle, such as 2 to 20 mol %, such as 4 to 36 mol %, preferably 12 to 20 mol % preferably 20 to 31 mol %,
at least one lysobisphosphatidic acid in an amount in the range from 0 to 12 mol %, such as from 4 to 12 mol %, more preferably in an amount from 4 to 6 mol %, more preferably in an amount from 10 to 12 mol %
less than 5 mol % of diacylglycerols,
less than 5 mol % of triacylglycerols, and
less than 5 mol % of phosphatidylinositols,
wherein all mol % amounts are calculated based on the amount of the unloaded lipid vesicle [in mol]. In a preferred embodiment the lipid vesicle comprises phosphatidylethanolamine(s) and cholesterol in a total amount from 2 to 90 mol %, preferably from 30 to 80 mol %, more preferably from 31 to 65 mol %, of each lipid vesicle based on the total amount of the unloaded lipid vesicle as such in mol and is calculated as the sum of phosphatidylethanolamine(s) and cholesterol.

In one embodiment the lipids vesicle comprises or consists of all the lipids in the following ranges:
cholesterol in an amount in the range from 10 to 15 mol %, such as from 12 to 14.5 mol %,
at least one sphingomyelin in an amount in the range from 2 to 8 mol %, such as 3 to 5 mol %,
at least one phosphatidylcholine in an amount in the range from 25 to 30 mol %, such as from 26 to 29 mol %,
at least one phosphatidylethanolamines in an amount in the range from 10 to 25 mol %, such as from 15 to 19.5 mol %,
at least one phosphatidylserine in an amount in the range from 12 to 20 mol %, Such as 12 to 14.5 mol %,
at least one lysobisphosphatidic acid in an amount in the range from 3 to 5 mol %
at least one diacylglycerol in an amount in the range from 3 to 5 mol %,
at least one triacylglycerol in the range from 3 to 6 mol %, and
at least one phosphatidylinositols in the range from 3 to 5 mol %,
wherein all mol % amounts are calculated based on the amount of the unloaded lipid vesicle [in mol]. Preferably, the lipid vesicle comprises or consists of, the lipids in the above described preferred ranges, cholesterol, L-alpha-phosphatidylethanolamine, L-alpha PS, at least one phosphatidylcholine, dipalmitin, glycerol tristearate, L-alpha-phosphatidylinositol and bis(monooleoylglycero)phosphate, and wherein the sphingomyelin is selected from the group consisting of milk sphingomyelin, brain sphingomyelin, egg sphingomyelin and mixtures thereof, and wherein the phosphatidylcholine is egg PC and/or DOPC.

According to a second embodiment, the lipid vesicle comprises or consists of, cholesterol, at least one sphingomyelin, at least one phosphatidylcholine, at least one phosphatidylethanolamine and least one phosphatidylserine. In a preferred embodiment the lipid vesicle comprises or consists of:
cholesterol in an amount in the range from 10 to 25 mol %, preferably in the range from 14 to 22 mol %, more preferably in the range from 17 to 19 mol %,
the at least one sphingomyelin in an amount in the range from 4 to 10 mol %, preferably in the range from 4 to 8 mol %, more preferably in the range from 5 to 6 mol %,
the at least one phosphatidylcholine in an amount in the range from 10 to 40 mol %, preferably in the range from 10 to 37 mol %, more preferably in the range from 30 to 36 mol %, preferably the phosphatidylcholine is selected from EG PC, DLPC and/or DOPC,
the at least one phosphatidylethanolamines in an amount of 10 to 30 mol %, preferably in the range from 15 to 25 mol %, more preferably in the range from 20 to 24 mol %,
the at least one phosphatidylserine in an amount in the range from 10 to 20 mol %, more preferably in the range from 15 to 18 mol %,
wherein all mol % amounts are calculated based on the amount of the unloaded lipid vesicle [in mol]. Preferably, the lipid vesicle comprises, in the above described preferred ranges, cholesterol, L-alpha-phosphatidylethanolamine, at least one sphingomyelin, at least one phosphatidylcholine and L-alpha PS, and wherein the sphingomyelin is selected from the group consisting of milk sphingomyelin, brain sphingomyelin, egg sphingomyelin and mixtures thereof, and wherein the phosphatidylcholine is egg PC and/or DOPC.

In another preferred embodiment of the second embodiment the lipid vesicle comprises or consists of:
at least cholesterol in an amount in the range from 4 to 14.5 mol %, or from 16 to 19 mol %, or from 30 to 50 mol %,
at least one sphingomyelin in an amount in the range from 2 to 24, such as from 3 to 4.8 mol %, or from 5.9 to 24 mol %, or from 35 to 45 mol %,
at least one phosphatidylcholine in an amount in the range from 1.5 to 29 mol %, such as 12 to 29 mol %, or from 32 to 36 mol %,
at least one phosphatidylethanolamines in an amount in the range from 2.5 to 3 mol %, or from 10 to 20 mol %, such as from 11 to 19 mol %, or from 22 to 25 mol %, such as from 23 to 25 mol %, or from 30 to 40 mol %, or from 44 to 70 mol %, and
at least one phosphatidylserine in an amount in the range from 2 to 3 mol % or from 10 to 20 mol %, such as 10 to 14.5 mol %, such as 16 to 18 mol %, or from 25 to 45 mol %,
wherein all mol % amounts are calculated based on the amount of the unloaded lipid vesicle [in mol]. Preferably, the lipid vesicle comprises, in the above described preferred ranges, cholesterol, L-alpha-phosphatidylethanolamine, at least one sphingomyelin, at least one phosphatidylcholine and L-alpha PS, and wherein the sphingomyelin is selected from the group consisting of milk sphingomyelin, brain sphingomyelin, egg sphingomyelin and mixtures thereof, and wherein the phosphatidylcholine is egg PC and/or DOPC. Preferably, the lipid vesicle of the embodiment comprises phosphatidylethanolamine(s) and cholesterol in a total amount from 2 to 90 mol %, preferably from 30 to 80 mol %, more preferably from 31 to 65 mol %, of each lipid vesicle based on the total amount of the unloaded lipid vesicle as such in mol and is calculated as the sum of phosphatidylethanolamine(s) and cholesterol.

According to a third preferred embodiment, the lipid vesicle comprises or consists of cholesterol, at least one sphingomyelin, at least one phosphatidylcholine, at least one phosphatidylethanolamine and least one phosphatidylserine and at least one lysobisphosphatidic acid. According to this embodiment, the lipid vesicle preferably comprises or consists of:
cholesterol in an amount in the range from 10 to 25 mol %, preferably in the range from 15 to 22 mol %, more preferably in the range from 15 to 18 mol %,
at least one sphingomyelin in an amount in the range from 4 to 10 mol %, preferably in the range of 4 to 13 mol %, more preferably in the range from 5 to 6 mol %,
at least one phosphatidylcholine in an amount in the range from 15 to 40 mol %, preferably in the range from 20 to 37 mol %, more preferably in the range from 25 to 35 mol %, more preferably in the range from 31 to 34 mol %,
at least one phosphatidylethanolamines in an amount of 15 to 45 mol %, preferably in the range from 18 to 25 mol %, more preferably in the range from 20 to 23 mol %,
at least one phosphatidylserine in an amount of 10 to 30 mol %, preferably in the range from 12 to 20 mol %, more preferably in the range from 15 to 18 mol %,
at least one lysobisphosphatidic acid in an amount of less than 7 mol %, preferably in the range from 3 to 6 mol %,
wherein all mol % amounts are calculated based on the amount of the unloaded lipid vesicle [in mol]. Preferably, the lipid vesicle comprises or consists of, in the above described preferred ranges, cholesterol, L-alpha-phosphatidylethanolamine, at least one sphingomyelin, at least one phosphatidylcholine, bis(monooleoylglycero)phosphate and L-alpha PS, and wherein the phosphatidylcholine is egg PC and/or DOPC, and wherein the sphingomyelin is selected from the group consisting of milk sphingomyelin, brain sphingomyelin, egg sphingomyelin and mixtures thereof, preferably milk sphingomyelin.

According to a fourth preferred embodiment, the lipid vesicle comprises or consists of, cholesterol, at least one sphingomyelin, at least one phosphatidylethanolamine and at least one lysobisphosphatidic acid. According to this embodiment, the lipid vesicle preferably comprises or consists of:
cholesterol in an amount in the range from 10 to 50 mol %, preferably in the range from 20 to 38 mol %, more preferably in the range from 31 to 34 mol %, or in the range from 25 to 38 mol %,
at least one sphingomyelin in an amount in the range from 4 to 15 mol %, preferably in the range from 8 to 13 mol %, more preferably in the range from 10 to 13 mol %,
at least one phosphatidylethanolamines in an amount of 20 to 50 mol %, preferably in the range from 40 to 50 mol %, more preferably in the range from 43 to 40 mol %,
at least one lysobisphosphatidic acid in an amount of 0 to 12 mol %, preferably 1 to 9 mol %, preferably 3 to 7 mol %, preferably 4 to 12 mol %, preferably in the range from 10 to 12 mol %,
wherein all mol % amounts are calculated based on the amount of the unloaded lipid vesicle [in mol]. Preferably, the lipid vesicle comprises or consists of, in the above described preferred ranges, cholesterol, L-alpha-phosphatidylethanolamine, at least one milk sphingomyelin, and bis(monooleoylglycero)phosphate, and wherein the sphingomyelin is selected from the group consisting of milk sphingomyelin, brain sphingomyelin, egg sphingomyelin and mixtures thereof, preferably milk sphingomyelin. Preferably, the lipid vesicle of the embodiment comprises phosphatidylethanolamine(s) and cholesterol in a total amount from 2 to 90 mol %, preferably from 30 to 80 mol %, more preferably from 31 to 65 mol %, of each lipid vesicle based on the total amount of the unloaded lipid vesicle as such in mol and is calculated as the sum of phosphatidylethanolamine(s) and cholesterol.

According to a fifth embodiment, the lipid vesicle comprises or consists of, cholesterol, at least one sphingomyelin, at least one phosphatidylcholine, at least one phosphatidylethanolamine and least one phosphatidylserine. According to this embodiment, the lipid vesicle preferably comprises or consists of:
cholesterol in an amount in the range from 40 to 55 mol %, preferably in the range from 45 to 50 mol %,
at least one sphingomyelin in an amount in the range from 10 to 15 mol %, such as from 12 to 15 mol %,
at least one phosphatidylcholine in an amount in an amount in the range from 10 to 15 mol %, such as from 12 to 15 mol %,
at least one phosphatidylethanolamines in an amount in the range from 10 to 15 mol %, such as from 12 to 15 mol %, and
at least one phosphatidylserine in an amount in the range from 10 to 15 mol %, such as from 12 to 15 mol %,
   wherein all mol % amounts are calculated based on the amount of the unloaded lipid vesicle [in mol]. Preferably, the lipid vesicle comprises, in the above described preferred ranges, cholesterol, L-alpha-phosphatidylethanolamine, at least one sphingomyelin, at least one phosphatidylcholine and L-alpha PS, and wherein the sphingomyelin is selected from the group consisting of milk sphingomyelin, brain sphingomyelin, egg sphingomyelin and mixtures thereof, and wherein the phosphatidylcholine is preferably egg PC and/or DOPC.

According to a sixth embodiment, the lipid vesicle comprises or consists of, cholesterol, at least one sphingomyelin, at least one phosphatidylcholine, at least one phosphatidylethanolamine and least one phosphatidylserine. According to this embodiment the lipid vesicle preferably comprises or consists of:
cholesterol in an amount in the range from 2 to 10 mol %, preferably in the range from 4 to 6 mol %,
at least one sphingomyelin in an amount in the range from 2 to 10 mol %, preferably in the range from 4 to 5 mol %;
at least one phosphatidylcholine in an amount in an amount in the range from 2 to 10 mol %, preferably in the range from 4 to 6 mol %,
at least one phosphatidylethanolamines in an amount in the range from 60 to 75, such as from 68 to 72 mol %,
at least one phosphatidylserine in an amount in the range from 10 to 20 mol %, such as in the range from 16 to 18 mol %
   wherein all mol % amounts are calculated based on the amount of the unloaded lipid vesicle [in mol]. Preferably, the lipid vesicle comprises, in the above described preferred ranges, cholesterol, L-alpha-phosphatidylethanolamine, at least one sphingomyelin, at least one phosphatidylcholine and L-alpha PS, and wherein the sphingomyelin is selected from the group consisting of milk sphingomyelin, brain sphingomyelin, egg sphingomyelin and mixtures thereof, and wherein the phosphatidylcholine is preferably egg PC and/or DOPC.

It has been shown in the studies in the present invention that—after oral administration of the lipid vesicle of the invention—a nucleic acid molecule comprised by the lipid vesicle as set forth herein was delivered to the central nervous system, spleen, gastrointestinal tract, liver and T-cells (see Examples section).

Accordingly, the invention provides a lipid vesicle as set forth herein carrying a nucleic acid molecule for use as a medicament, wherein said lipid vesicle is administered orally, wherein said nucleic acid molecule is delivered to one or more of the target tissues selected from the group consisting of the central nervous system, spleen, gastrointestinal tract, liver and T-cells.

The present invention provides a method of delivery of a nucleic acid molecule to the central nervous system, spleen, gastrointestinal tract, liver and T-cells that allows for an efficient modulation of the target nucleic acid in these cells/tissues following oral administration.

The invention provides a lipid vesicle as defined herein carrying a nucleic acid molecule for use as a medicament, wherein said lipid vesicle is administered orally, wherein expression of a target nucleic acid in one or more of the target tissues selected from the group consisting of the central nervous system, spleen, gastrointestinal tract, liver and T-cells is modulated.

The term “nucleic acid molecule” has been defined above. In an embodiment, the nucleic acid molecule is an antisense oligonucleotide. In another embodiment, the nucleic acid molecule is an RNAi molecule.

In one embodiment the nucleic acid molecule comprised by the lipid vesicles provided herein is a modified siRNA with at least one backbone modification selected from a modified internucleoside linkage and/or a modified sugar nucleoside.

Preferably, the oligonucleotide is an antisense oligonucleotide, such as a single-stranded antisense oligonucleotide. The single-stranded antisense oligonucleotide may be a modified single-stranded antisense oligonucleotide.

In one embodiment the nucleic acid molecule comprised by the lipid vesicles provided herein is a modified single-stranded antisense oligonucleotide with at least one backbone modification selected from a modified internucleoside linkage and/or a modified sugar nucleoside.

In one embodiment the modified single-stranded antisense oligonucleotide comprised by the lipid vesicle provided herein is a single-stranded antisense oligonucleotide with at least one modified internucleoside linkage. In an embodiment, at least 50%, such as at least 75% of the internucleoside linkages in the antisense oligonucleotide are modified internucleoside linkages. Further, it is envisaged that all internucleoside linkages of said single-stranded antisense oligonucleotide are modified internucleoside linkages.

In one embodiment the modified internucleoside linkage are selected from the group comprising phosphorothioate, diphosphorothioate, phosphordiamidates and boranophosphate linkages. In particular, the modified internucleoside linkages are phosphorothioate linkages. In one embodiment all the internucleoside linkages are phosphorothioate linkages.

In one embodiment the modified single-stranded antisense oligonucleotide comprised by the lipid vesicle provided herein is a single-stranded antisense oligonucleotide with at least one sugar modification in the backbone. The sugar modification can for example be selected from morpholino, PNA or 2′ sugar modified nucleosides.

In one embodiment, the modified single-stranded antisense oligonucleotide is a single-stranded morpholino antisense oligonucleotide. In another embodiment, the modified single-stranded antisense oligonucleotide is single-stranded peptide nucleic acids (PNA).

In a preferred embodiment, the modified single-stranded antisense oligonucleotide comprises or consists of at least one modified internucleoside linkage and one or more 2′ sugar modified nucleosides. It is advantageous if the single-stranded antisense oligonucleotide comprises at least three 2′ sugar modified nucleosides, in particular located in the 5′ and 3′ terminal ends of the oligonucleotide or contiguous nucleotide sequence thereof. The one or more 2′ sugar modified nucleosides can independently be selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA (locked nucleic acid) nucleosides. In a preferred embodiment, the modified single-stranded antisense oligonucleotide, is a single-stranded antisense oligonucleotides with at least one modified internucleoside linkage and comprises one or more LNA (locked nucleic acid) nucleosides. Accordingly, the modified single-stranded antisense oligonucleotide is preferably a single-stranded antisense LNA oligonucleotide with at least one modified internucleoside linkage, such as at least 50%, such as alt least 75% phosphorothioate nucleoside linkages.

The modified single-stranded antisense oligonucleotide with at least one 2′ sugar modified nucleoside and preferably at least one modified internucleoside linkage, is selected from the group of gapmers, mixmers, totalmers, antimiRs, blockmiRs and splice-switching oligonucleotides (SSOs). Thus, the modified single-stranded antisense oligonucleotide may be a gapmer as defined herein (such as a LNA gapmer). Alternatively, the modified single-stranded antisense oligonucleotide may be a mixmer as defined herein. Alternatively, the modified single-stranded antisense oligonucleotide may be totalmer as defined herein. Alternatively, the modified single-stranded antisense oligonucleotide may be an antimiR as defined herein. Alternatively, the modified single-stranded antisense oligonucleotide may be a splice-switching oligonucleotide as defined herein.

In particular, it is envisaged that the modified single-stranded antisense oligonucleotide is an LNA antisense oligonucleotide.

The modified single-stranded antisense oligonucleotide, e.g. the backbone modified single-stranded antisense oligonucleotide, to be loaded into the lipid vesicle provided herein typically have a length of 7 to 35 nucleotides, such as 7 to 30 nucleotides. They may be in the form of a pharmaceutically acceptable salt.

In an embodiment, the modified single-stranded antisense oligonucleotide comprises a contiguous nucleotide sequence of 7 to 35 nucleotides, such as 7 to 30 nucleotides, wherein the contiguous nucleotide sequence is at least 90% complementary, such as fully complementary, to a target nucleic acid in the target tissue.

In an embodiment, the modified single-stranded antisense oligonucleotide comprises a contiguous nucleotide sequence of at least 7 to 26 nucleotides, wherein the contiguous nucleotide sequence is at least 90% complementary, such as fully complementary to a target nucleic acid in the target tissue.

In an embodiment, the modified single-stranded antisense oligonucleotide comprises a contiguous nucleotide sequence of 10 to 30 nucleotides such as of 7 to 14 nucleotides or of 14 to 20 nucleotides, wherein the contiguous nucleotide sequence is at least 90% complementary, such as fully complementary to a target nucleic acid in the target tissue.

In an embodiment wherein the single-stranded antisense oligonucleotide is conjugated to a lipophilic conjugate moiety. Preferably the single-stranded antisense oligonucleotide includes a biocleavable nucleotide linker in the 5′ terminal or 3′ terminal of the oligonucleotide to which the lipophilic conjugate moiety is covalently attached.

In one embodiment the single stranded antisense oligonucleotide comprises a biocleavable nucleotide region (region D′ and/or D″) positioned between the contiguous nucleotide sequence of the gapmer, mixmer or totalmer and a lipophilic conjugate moiety, such as a cholesterol or tocopherol conjugate moiety.

The lipid vesicle herein shall carry, i.e. comprise, the nucleic acid molecule referred to herein (such as the modified single-stranded oligonucleotide or RNAi molecule). Accordingly, the nucleic acid molecule is encapsulated in or adsorbed to the lipid vesicles. In other words, the lipid vesicle provided herein is loaded with the nucleic acid molecule. In a further embodiment the lipid vesicle is formulated for oral delivery.

The lipid vesicle comprises an amount of the nucleic acid molecule which allows for the modification of the target nucleic acid (in particular in the target tissue/target cell). The lipid vesicle carrying the nucleic acid molecule may also be said to comprise a payload of the nucleic acid molecule. Thus, the lipid vesicle may comprise an amount of the nucleic acid that allows for modulating the expression of the target gene.

The lipid vesicle carrying the nucleic acid molecule such as the antisense oligonucleotide or RNAi oligonucleotide as referred to herein is administered orally. Preferably, a pharmaceutical effective amount of the lipid vesicle carrying the nucleic acid molecule is administered. Oral administration allows for delivering the lipid vesicle, and thus the payload of the nucleic acid molecule into target tissues selected from the group consisting of the central nervous system, spleen, gastrointestinal tract, liver and T-cells.

The disease to be treated in accordance with the present invention is associated with expression of the target nucleic acid or can be treated by changing the expression of the target nucleic acid. Accordingly, the disease can be caused by abnormal levels of the target nucleic acid. In a preferred embodiment, the disease is associated with expression of the target nucleic acid in the central nervous system, in the spleen, in the gastrointestinal tract, in the liver and/or in T-cells. Accordingly, the disease can be caused by abnormal levels of the target nucleic acid in the central nervous system, in the spleen, in the gastrointestinal tract, in the liver and/or in T-cells. In a preferred embodiment, the disease is a disease that can be treated by reduction of the level of the target nucleic acid, in particular in the central nervous system, in the spleen, in the gastrointestinal tract, in the liver and/or in T-cells. In a preferred embodiment, the disease is a disease that can be treated by slice switching of target nucleic acid to increase the amount of functional protein expressed from the target nucleic acid, in particular in the central nervous system, in the spleen, in the gastrointestinal tract, in the liver and/or in T-cells.

In an embodiment, the nucleic acid molecule is delivered to the central nervous system. The term “central nervous system” preferably encompasses the brain and the spinal cord.

Specific tissues in the brain, to which the single stranded antisense oligonucleotide may be delivered, encompass the cerebellum, cerebral cortex, mid brain, thalamus, hypothalamus, hippocampus, striatum, frontal temporal lobes, motor cortex and the brain stem. Specific tissues in the spinal cord are basal root ganglion, dorsal horn and ventral horns. Accordingly, the modified single-stranded antisense oligonucleotide is delivered to the brain, the spinal cord, or both to the brain and spinal cord or specific tissues thereof.

Preferably, the delivery to the central nervous system (such as to the brain) allows for the treatment of a disease selected from the group consisting of brain cancer (such as a brain tumor), a seizure disorder, a neurodegenerative disorder, a neuropsychiatric disorder and a movement disorders. More preferably, the delivery to the central nervous system (such as to the brain) allows for the treatment of a disease selected from Angelman disorder, Alexander Disease, Alzheimer's disease, Amyotrophic lateral sclerosis, Friedreich's ataxia, Huntington's disease, Lewy body disease, Parkinson's disease, Spinal muscular atrophy, Schizophrenia, Depression, Bipolar disease, Autism, Epilepsy, Frontotemporal dementia, Progressive Bulbar Palsy, progressive supranuclear palsy, Rett syndrome, Tourette syndrome, Neurofibromatosis, progressive muscular atrophy, hereditary spastic paraplegia, Pelizaeus-Merzbacher disease, Gaucher's disease, Spinocerebellar ataxia, Pagon Bird Detter syndrome; Friedreich's ataxia; Spinocerebellar ataxia, Digeorge syndrome, Dup15q syndrome, Doose Syndrome, Glut1 Deficiency Syndrome, CDKL5 Disorder, Frontal Lobe Epilepsy, Childhood Absence Epilepsy, Early Myoclonic Encephalopathy (EME), Lennox-Gastaut Syndrome (LGS), Ohtahara Syndrome, and Landau-Kleffner Syndrome. Accordingly, the aforementioned diseases can be treated by oral administration of the lipid vesicle as referred to herein.

In an embodiment, the nucleic acid molecule is delivered to the spleen.

Preferably, the delivery to the spleen allows for the treatment of a disease selected from the group consisting of cancer such as spleen cancer, an inflammatory disease and an immunodisorder. Accordingly, the aforementioned diseases can be treated by oral administration of the lipid vesicle as referred to herein.

In an embodiment, the nucleic acid molecule is delivered to the liver.

Preferably, the delivery to the liver allows for the treatment of a disease selected from the group consisting of liver cancer, cardiovascular diseases, coagulation cascade deficiencies, inflammatory diseases, metabolic disease and infections. More preferably, the delivery to the liver allows for the treatment of a disease selected from hepatocellular carcinoma, liver malignancies metastasized from primary cancers in other tissues, diabetes type 1, non-insulin dependent diabetes, insulin resistance, control of blood glucose levels, HDL/LDL cholesterol imbalance, atherosclerosis, dyslipidemias, familial combined hyperlipidemia (FCHL), acquired hyperlipidemia, statin-resistant hypercholesterolemia, cardiovascular disease, coronary artery disease (CAD), and coronary heart disease (CHD), non-alcoholic steatohepatitis (NASH), non-alcoholic fatty liver disease, obesity, acute coronary syndrome (ACS), thrombosis, rare bleeding disorders, hepatitis B or C, cytomegalovirus infection, schistosomiasis infection and leptospirosis infection, malaria, fasciola infection, rheumatoid arthritis, liver fibrosis, cirrhosis, hepatic porphyria, acute intermittent porphyria (AIP), paroxysmal nocturnal hemoglobinuria (PNH), atypical hemolytic-uremic syndrome (aHUS), myasthenia gravis, neuromyelitis optica, alpha 1-antitrypsin deficiency, Cushing's Syndrome, and transthyretin-related hereditary amyloidosis. Accordingly, the aforementioned diseases can be treated by oral administration of the lipid vesicle as referred to herein.

In an embodiment, the nucleic acid molecule is delivered to T-cell.

Preferably, the delivery to T-cells allows for the treatment of a disease selected from the group consisting of cancer, an inflammatory disease and an infection disease.

In an embodiment, the nucleic acid molecule is delivered to the gastrointestinal tract. The gastrointestinal tract is the part of the digestive system that comprises the stomach and the intestines. Thus, the nucleic acid molecule may be delivered to the stomach and/or the intestines, i.e. the small or large intestine. Preferably, the delivery to the gastrointestinal tract allows for the treatment of a colorectal cancer gastric cancer, metabolic disorders or inflammatory bowel disease (such as Crohn's disease or ulcerative colitis).

In a further aspect, the invention provides a pharmaceutical composition comprising the lipid vesicle provided herein which carries a nucleic acid such as an oligonucleotide (e.g. a single-stranded antisense oligonucleotide as defined elsewhere herein). Said pharmaceutical composition may further comprise a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In particular, the pharmaceutical composition is a formulation suitable for oral administration

The invention provides methods for treating a disease by oral administration of a therapeutically effective amount of a nucleic acid molecule comprised (e.g. formulated, encompassed, encapsulated, loaded) in the lipid vesicle provided herein as referred to above or the pharmaceutical composition of the invention to a subject suffering from the disease.

The invention provides methods for treating a disease in the central nervous system, spleen, gastrointestinal tract, liver and T-cells by oral administration of a therapeutically effective amount of a nucleic acid molecule comprised (e.g. formulated, encompassed, encapsulated, loaded) in the lipid vesicle provided herein as referred to above or the pharmaceutical composition of the invention to a subject suffering from the disease. Preferred diseases are mentioned above. Upon oral administration, the nucleic acid molecule such as the modified single-stranded antisense oligonucleotide is delivered to one or more of the target tissues selected from the group consisting of the central nervous system, spleen, gastrointestinal tract, liver and T-cells.

The invention also provides for the use of the lipid vesicle comprising a nucleic acid molecule such as a modified single stranded antisense oligonucleotide as described above for the manufacture of a medicament for the treatment of a disease. Preferred diseases are mentioned above. The definitions and explanations given above apply accordingly. Preferably, the lipid vesicle is administered orally, thereby delivering the nucleic acid molecule to one or more of the target tissues selected from the group consisting of the central nervous system, spleen, gastrointestinal tract, liver and T-cells.

Method of Manufacture

In a further aspect, the invention provides methods for manufacturing the lipid vesicles carrying, i.e. comprising the nucleic acid molecule as referred to herein in connection with the present invention. The definitions above apply accordingly.

The lipid vesicles according to the present invention are preferably prepared using a thin-film rehydration method. This method preferably comprises the steps
forming a solution in an organic solvent of at least one lipid vesicle-forming component
removing the solvent thereby forming a lipid film
rehydrating the film in an aqueous solvent, preferably a buffer, in the presence of the nucleic acid molecule, such as an antisense oligonucleotide or RNAi molecule,
thereby forming the lipid vesicle carrying the nucleic acid molecule.

The organic solvent is preferably selected from the group consisting of methanol, chloroform, and dichloromethane, ethanol, dichloromethane, as well as isopropanol and mixtures of two or more thereof. As aqueous solvent, preferably an aqueous buffer is employed.

The present invention thus also relates to the above described method, as well as to lipid vesicles obtained or obtainable by said method.

### LIST OF EMBODIMENTS

In the following especially preferred embodiments of the present invention are described:
1. A lipid vesicle carrying a nucleic acid molecule for use as a medicament, wherein the lipid vesicle has a hydrodynamic diameter Dₕ of less than 300 nm, measured according to DLS, preferably a diameter in the range of 50 to 300 nm, wherein said lipid vesicle is administered orally, wherein said nucleic acid molecule is for delivery to one or more of the target tissues selected from the group consisting of the central nervous system, spleen, gastrointestinal tract, liver and T-cells.
2. The lipid vesicle for the use according to embodiment 1, wherein the lipid vesicle has a hydrodynamic diameter Dₕ of less than 200 nm, measured according to DLS, preferably a diameter in the range from 50 to 250 nm.
3. The lipid vesicle for the use according to embodiment 1 or 2, wherein the lipid vesicle has a hydrodynamic diameter Dh of less than 180 nm, measured according to DLS, preferably a diameter in the range from 60 to 180 nm, such as from 100 to 160 nm.
4. The lipid vesicle for the use according to any one of embodiments 1 to 3, wherein the lipid vesicle is stable in gastric fluid at a temperature of 20° C. for at least 5 h.
5. The lipid vesicle for the use according to any one of embodiments 1 or 4, wherein the lipid vesicles are stable in fasted state intestinal fluid at a temperature of 20° C. for at least 5 h.
6. The lipid vesicle for the use according to embodiment 4 or 5, where the hydrodynamic diameter of the lipid vesicle changes at most by 90% after 5 h incubation in gastric fluid when compared to the respective initial Dh measured before incubation.
7. The lipid vesicle for the use according to embodiment 6, wherein the hydrodynamic diameter of the lipid vesicle changes at most by 60% and the Dh of the lipid vesicle is in the range from 80 to 180 nm both a time 0 and 5 h after incubation.
8. The lipid vesicle for the use according to embodiment 4 to 7 where the hydrodynamic diameter of the lipid vesicle changes at most by 40% after 5 h incubation in intestinal fluid when compared to the respective initial Dh measured before incubation.
9. The lipid vesicle for the use according to embodiment 8, wherein the hydrodynamic diameter of the lipid vesicle changes at most by 25% and the Dh of the lipid vesicle is in the range from 80 to 180 nm both a time 0 and 5 h after incubation.
10. The lipid vesicle for the use according to any one of embodiments 1 to 9, wherein the lipid vesicle comprises cholesterol and at least one phosphatidylethanolamine.
11. The lipid vesicle for the use according to embodiment 10, wherein the phosphatidylethanolamine is an L-alpha-phosphatidyl-ethanolamine.
12. The lipid vesicle for the use according to any one of embodiments 1 to 11, wherein the lipid vesicle comprises cholesterol in an amount of 4 to 50 mol %, based on the total amount of the unloaded lipid vesicle (in mol).
13. The lipid vesicle for the use according embodiment 12, wherein the lipid vesicle comprises cholesterol in an amount from 4 to 14.5 mol %, or from 16 to 19 mol % or from 30 to 50 mol % based on the total amount of the unloaded lipid vesicle (in mol).
14. The lipid vesicle for the use according to any one of embodiments 1 to 13, wherein the lipid vesicle comprises L-alpha-phosphatidylethanolamine in an amount of 2 to 70 mol %, based on the total amount of the unloaded lipid vesicle (in mol).
15. The lipid vesicle for the use according embodiments 14, wherein the lipid vesicle comprises L-alpha-phosphatidylethanolamine in an amount from 11 to 19 mol %, or from 23 to 25 mol % or from 30 to 40 mol % or from 44 to 70 mol % based on the total amount of the unloaded lipid vesicle (in mol)
16. The lipid vesicle for the use according to any one of embodiments 1 to 15, wherein the lipid vesicle comprises at least one phosphatidylethanolamine, preferably L-alpha-phosphatidyl-ethanolamine, and cholesterol in a total amount from 2 to 90 mol %, preferably from 30 to 80 mol %, based on the total amount of the unloaded lipid vesicle (in mol).
17. The lipid vesicle for the use according any one of embodiments 10 to 16, wherein the lipid vesicle further comprises at least one phosphatidylcholine and/or at least one phosphatidylserine and/or at least one lysobisphosphatidic acid.
18. The lipid vesicle for the use according embodiments 17, wherein
   i. the phosphatidylcholine is in an amount from 0 to 40 mol %, such as 1.5 to 29 mol %, such as 12 to 29 mol % such as 32 to 36 mol % and/or
   ii. the at least one phosphatidylserine is in an amount from 0 to 45 mol %, such as from 2 to 3 mol %, such as from 10 to 20 mol % such as from 25 to 45 mol %, and/or
   iii. the at least one lysobisphosphatidic acid is in an amount from 0 to 12 mol %, such as from 4 to 12 mol %, such as from 10 to 12 mol %.
19. The lipid vesicle for the use according to embodiment 17 or 18, wherein the total amount of phosphatidylcholine, phosphatidylserine and lysobisphosphatidic acid in the lipid vesicle is in the range from 10 to 54 mol %, based on the total amount of the unloaded lipid vesicle (in mol).
20. The lipid vesicle for the use according embodiment 17 or 19, wherein the at least one phosphatidylcholine is Egg-PC, DLPC or DOPC.
21. The lipid vesicle for the use according embodiment 17 or 20, wherein the at least one phosphatidylcholine is Egg-PC or DOPC.
22. The lipid vesicle for the use according to any one of embodiments 10 to 21, wherein the lipid vesicle further comprises at least one sphingomyelin, in particular a sphingomyelin selected from the group consisting of milk sphingomyelin, brain sphingomyelin, egg sphingomyelin and mixtures of two or more thereof.
23. The lipid vesicle for the use according to embodiment 22, wherein the lipid vesicle comprises the at least one sphingomyelin in an amount in the range from 2 to 45 mol %, such as from 2 to 24 mol %, such as from 3 to 4.8 mol %, such as from 4 to 15 mol %, such as from 5.9 to 24 mol % such as from 35 to 45 mol % based on the total amount of the unloaded lipid vesicle (in mol).
24. The lipid vesicle for the use according to any one of embodiments 10 to 23, wherein the lipid vesicle optionally further comprises at least one diacylglycerol and/or at least one triacylglycerol and/or at least one phosphatidylinositol, wherein the total amount of the sum of diacylglycerols and triacylglycerols and phosphatidylinositols is in the range from 0 to 15 mol %, based on the total amount of the unloaded lipid vesicle (in mol).
25. The lipid vesicle for the use according to embodiment 24, wherein the lipid vesicle comprises
   i. the diacylglycerols in an amount from 0 to 5 mol %, such as from 3 to 5 mol %;
   ii. the triacylglycerols in an amount from 0 to 5 mol %, such as from 3 to 5 mol %; and
   iii. the phosphatidylinositols in an amount from 0 to 5 mol %, such as from 3 to 5 mol %,
      and wherein the total amount of diacylglycerols and triacylglycerols and phosphatidylinositols is in the range from 4 to 15 mol %, each based on the total amount of the unloaded lipid vesicle (in mol).
26. The lipid vesicle for the use according to embodiment 1 to 25, wherein the lipid vesicle has a hydrodynamic diameter Dₕ of 50 to 250 nm and comprises
   i. cholesterol in an amount of 4 to 50 mol %, based on the total amount of the unloaded lipid vesicle (in mol);
   ii. at least one phosphatidylethanolamine in an amount in the range from 10 to 70 mol % based on the total amount of the unloaded lipid vesicle (in mol), wherein the lipid vesicle,
   iii. at least one sphingomyelin in an amount in the range from 4 to 15 mol %, based on the total amount of the unloaded lipid vesicle (in mol), wherein the lipid vesicle; and
   iv. optionally comprises at least one diacylglycerol and/or at least one triacylglycerol and/or at least one phosphatidylinositol, wherein the total amount of the sum of diacylglycerols and triacylglycerols and phosphatidylinositols is in the range from 0 to 15 mol %, such as from 4 to 15 mol %, based on the total amount of the unloaded lipid vesicle.
27. The lipid vesicle for the use according to embodiment 26, wherein the lipid vesicle further comprises phosphatidylcholine and/or phosphatidylserine and/or lysobisphosphatidic acid.
28. The lipid vesicle according to embodiment 26 or 27, wherein the phosphatidylcholine is Egg-PC or DOPC.
29. The lipid vesicle for the use according to embodiment 27 or 28, wherein the lipid vesicle comprises 4 to 36 mol % Egg-PC or 4 to 36 mol % DOPC based on the total amount of the unloaded lipid vesicle.
30. The lipid vesicle according to embodiment 26 to 29, wherein the phosphatidylserine is in the range from 12 to 20 mol % based on the total amount of the unloaded lipid vesicle.
31. The lipid vesicle according to embodiment 26 to 30, wherein the lysobisphosphatidic acid is in the range from 4 to 12 mol % based on the total amount of the unloaded lipid vesicle.
32. The lipid vesicle for the use according to embodiment 1 to 31, wherein the lipid vesicle has a hydrodynamic diameter Dₕ in the range from 60 to 180 nm and comprises,
   i. Cholesterol in an amount in the range from 10 to 15 mol %, such as from 12 to 14.5 mol %, based on the total amount of the unloaded lipid vesicle (in mol);
   ii. at least one phosphatidylethanolamine in the range from 10 to 25 mol %, such as from 15 to 19.5 mol %, based on the total amount of the unloaded lipid vesicle (in mol);
   iii. at least one milk sphingomyelin in the range from 2 to 8 mol %, such as from 3 to 5 mol %, based on the total amount of the unloaded lipid vesicle (in mol);
   iv. Egg-PC in the range from 25 to 30 mol %, such as from 26 to 29 mol %, based on the total amount of the unloaded lipid vesicle (in mol);
   v. at least one diacylglycerol in the range from 3 to 5 mol % based on the total amount of the unloaded lipid vesicle (in mol),
   vi. at least one triacylglycerol in the range from 3 to 5 mol % based on the total amount of the unloaded lipid vesicle (in mol),
   vii. at least one phosphatidylinositol in the range from 3 to 5 mol % based on the total amount of the unloaded lipid vesicle (in mol),
   viii. lysobisphosphatidic acid in the range from 3 to 6 mol % based on the total amount of the unloaded lipid vesicle (in mol); and
   ix. Phosphatidylserine in the range from 12 to 20 mol %, such as from 12 to 14.5 mol %, based on the total amount of the unloaded lipid vesicle (in mol).
33. The lipid vesicle for use according to embodiment 1, wherein the lipid vesicle is selected from F1, F3, F12, F13, F14, F17, F18, F20, F21, F22, F23, F24, F25, F26, F28, F30, F33 or F34.
34. The lipid vesicle for use according to embodiment 1 or 33, wherein the lipid vesicle is selected from compositions F1, F12, F13, F14, F17, F21, F22, F23, F25 or F33.
35. The lipid vesicle for use according to any one of embodiments 1 to 32, wherein the nucleic acid molecule is capable of modulating a target nucleic acid in the target tissue.
36. The lipid vesicle of use according to any one of embodiments 1 to 35, wherein nucleic acid molecule comprises at least one modified internucleoside linkage and/or a modified nucleoside.
37. The lipid vesicle of use according to embodiment 36, wherein the modified nucleoside is a 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA (locked nucleic acid) nucleosides
38. The lipid vesicle for use according to embodiment 36 or 37, wherein the modified internucleoside linkage is selected from the group consisting of phosphorothioate, diphosphorothioate and boranophosphate linkages.
39. The lipid vesicle for the use according to any one of embodiments 1 to 38, wherein the nucleic acid is an antisense oligonucleotide or an RNAi molecule.
40. The lipid vesicle for use according to embodiment 39, wherein the antisense oligonucleotide is a single-stranded oligonucleotide.
41. The lipid vesicle for use according to embodiment 39 or 40, wherein the antisense oligonucleotide 7 to 30 nucleotides in length, such as from 8 to 12 nucleotides, such as from 14 to 20 nucleotides.
42. The lipid vesicle for use according to embodiment 39 to 41, wherein the antisense oligonucleotide is selected from the group of gapmers, mixmers, totalmers, antimiRs, blockmiRs and splice-switching oligonucleotides.
43. The lipid vesicle for the use of embodiment 40 to 42, wherein at least 50%, such as at least 75%, of the internucleoside linkages in the antisense oligonucleotide are phosphorothioate linkages.
44. The lipid vesicle for the use of embodiment 43, wherein all internucleoside linkages of said single-stranded antisense oligonucleotide are phosphorothioate internucleoside linkages.
45. The lipid vesicle for the use of any one of embodiments 40 to 44, wherein the single-stranded antisense oligonucleotide comprises one or more modified nucleosides.
46. The lipid vesicle for the use of embodiment 45, wherein the modified nucleoside is a nucleoside where the ribose is replaced by a modification selected from the group consisting of a hexose ring (HNA), an unlinked ribose ring UNA, bicyclohexose, tricyclo nucleosides, peptide nucleic acids (PNA), or morpholino nucleic acids..
47. The lipid vesicle for the use of claim 45, wherein the modified nucleosides are 2′ sugar modified nucleosides such as 2′ sugar modified nucleosides independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA (locked nucleic acid) nucleosides.
48. The antisense oligonucleotide of embodiment 47, wherein the LNA nucleoside is selected from oxy-LNA, amino-LNA, thio-LNA, cET, and ENA.
49. The antisense oligonucleotide of embodiment 47 or 48, wherein the modified LNA nucleoside is oxy-LNA with the following 2′-4′ bridge —O—CH₂—.
50. The lipid vesicle for use according to embodiment 49, wherein the oxy-LNA is beta-D-oxy-LNA.
51. The lipid vesicle for use according to embodiment 42, wherein the antisense oligonucleotide is a gapmer of the formula 5′-F-G-F′-3′, where the F and F′ wing regions independently comprise or consist of 1-7 2′ sugar modified nucleosides in accordance with embodiment or 47 to 50 and G is a region between 5 and 18 nucleosides which are capable of recruiting RNaseH, such as DNA.
52. The lipid vesicle for the use of any one of claims 1 to 51, wherein
   a) said nucleic acid molecule is deliverable to the central nervous system such as to the brain and/or spinal cord, and wherein the medicament is for the treatment of a disease selected from the group consisting of brain cancer (such as a brain tumor), a seizure disorder, a neurodegenerative disorder, a neuropsychiatric disorder and a movement disorders, such as seizure disorder, neurodegenerative disorder, neuropsychiatric disorder or movement disorders is selected from the group consisting of Angelman disorder, Alexander Disease, Alzheimer's disease, Amyotrophic lateral sclerosis, Friedreich's ataxia, Huntington's disease, Lewy body disease, Parkinson's disease, Spinal muscular atrophy, Schizophrenia, Depression, Bipolar disease, Autism, Epilepsy, Frontotemporal dementia, Progressive Bulbar Palsy, progressive supranuclear palsy, Rett syndrome, Tourette syndrome, Neurofibromatosis, progressive muscular atrophy, hereditary spastic paraplegia, Pelizaeus-Merzbacher disease, Gaucher's disease, Spinocerebellar ataxia, Pagon Bird Detter syndrome; Friedreich's ataxia; Spinocerebellar ataxia, Digeorge syndrome, Dup15q syndrome, Doose Syndrome, Glut1 Deficiency Syndrome, CDKL5 Disorder, Frontal Lobe Epilepsy, Childhood Absence Epilepsy, Early Myoclonic Encephalopathy (EME), Lennox-Gastaut Syndrome (LGS), Ohtahara Syndrome, and Landau-Kleffner Syndrome,
   b) said nucleic acid molecule is deliverable to the spleen, and wherein the medicament is for the treatment of a disease selected from the group consisting of cancer such as spleen cancer, an inflammatory disease and an immunodisorder,
   c) said nucleic acid molecule is deliverable to T-cells, and wherein the medicament is for the treatment of a disease selected from the group consisting of cancer, an inflammatory disease and an infection disease, and/or
   d) said nucleic acid molecule is deliverable to the liver, and wherein the medicament is for the treatment of a disease selected from the group consisting of liver cancer, cardiovascular diseases, coagulation cascade deficiencies, inflammatory diseases, metabolic disease and infections, e.g. wherein the liver diseases are selected from the group consisting of hepatocellular carcinoma, liver malignancies metastasized from primary cancers in other tissues, diabetes type 1, non-insulin dependent diabetes, insulin resistance, control of blood glucose levels, HDL/LDL cholesterol imbalance, atherosclerosis, dyslipidemias, familial combined hyperlipidemia (FCHL), acquired hyperlipidemia, statin-resistant hypercholesterolemia, cardiovascular disease, coronary artery disease (CAD), and coronary heart disease (CHD), non-alcoholic steatohepatitis (NASH), non-alcoholic fatty liver disease, obesity, acute coronary syndrome (ACS), thrombosis, rare bleeding disorders, hepatitis B or C, cytomegalovirus infection, schistosomiasis infection and leptospirosis infection, malaria, fasciola infection, rheumatoid arthritis, liver fibrosis, cirrhosis, hepatic porphyria, acute intermittent porphyria (AIP), paroxysmal nocturnal hemoglobinuria (PNH), atypical hemolytic-uremic syndrome (aHUS), myasthenia gravis, neuromyelitis optica, alpha 1-antitrypsin deficiency, Cushing's Syndrome, and transthyretin-related hereditary amyloidosis.
53. A method for preparing a lipid vesicle carrying a nucleic acid molecule, wherein the lipid vesicle has a hydrodynamic diameter Dₕ of less than 300 nm, measured according to DLS, preferably a diameter in the range from 50 to 300 nm, wherein said lipid vesicle is for oral adiminstration, wherein said oligonucleotide is for delivery to one or more of the target tissues selected from the group consisting of the central nervous system, spleen, gastrointestinal tract, liver and T-cells, the method comprising the steps
   forming a solution in an organic solvent of at least one lipid vesicle-forming component
   removing the solvent thereby forming a lipid film
   rehydrating the film in an aqueous solvent, preferably a buffer, in the presence of the nucleic acid molecule,
   thereby forming the lipid vesicle carrying the nucleic acid molecule.
54. A lipid vesicle obtained or obtainable by the method according to embodiment 53.
55. The lipid vesicle according to embodiment 54, having the properties as described in embodiments 1 to 51.

### EXAMPLES

Materials and Methods

List of Antisense Oligonucleotides

TABLE 1
Oligonucleotides used:

| SEQ ID NO | CMP ID | Compound | Target |
|---|---|---|---|
| 1 | ASO1 | GAGttacttgccaACT | Malat 1 |

Capital letters represent beta-D-oxy LNA nucleosides, lowercase letters represent DNA nucleosides, all LNA C are 5-methyl cytosine, all internucleoside linkages are phosphorothioate internucleoside linkages unless specified, lowercase o indicate a phosphodiester internucleoside linkage

Analysis of Hydrodynamic Diameter

Mean hydrodynamic diameter of lipid vesicles was determined by dynamic light scattering (DLS) using a Zetasizer Ultra (Malvern Panalytical, Malvern, UK) at a laser wavelength of 685 nm. Scattered light was detected at an angle of 165°. Results are expressed as mean±SD of n=3 measurements in DPBS (No. 14190250, Thermo Scientific, Waltham, Mass., USA) at room temperature.

Analysis of Particle Morphology by Transmission Electron Microscopy

Particle morphology can be analyzed using transmission electron microscopy (TEM) after uranyl acetate negative staining. Formvar coated copper grids are glow discharged and incubated with 5 μL of lipid vesicle samples in DPBS (No. 14190250, Thermo Scientific, Waltham, Mass., USA) for 1 min. Samples are removed and grids are washed two times with ultra-pure water (No. 10977023, Thermo Scientific, Waltham, Mass., USA). Grids are then washed once using 2% uranyl acetate and are subsequently incubated in 2% uranyl acetate for 15 sec. Uranyl acetate is removed and grids are allowed to dry before analysis. Images are acquired using a JEM-1400 Plus transmission electron microscope (JEOL, Tokyo, Japan).

Zeta Potential by Dynamic and Electrophoretic Light Scattering

Zeta potential of lipid vesicles can be determined using a Zetasizer Ultra (Malvern Panalytical, Malvern, UK) according to the manufacturers' recommendations. Lipid vesicle F1 was measured in 0.1×DPBS at 25° C. Results are shown as mean±SD of n=3 measurements in table 6 in example 3 below. From these data the lipid vesicle F1 is characterized as monodisperse with a slight negative surface charge.

Loading Locked Nucleic Acid Antisense Oligonucleotide (LNA ASO) into Lipid Vesicles

LNA ASO was loaded by rehydrating the thin lipid film with LNA ASOs in DPBS (No. 14190250, Thermo Scientific, Waltham, Mass., USA).

The preparation of lipid vesicles was carried out as described above under the section “Lipid vesicle preparation” with addition of the desired amount of LNA-ASO in the rehydration step. Non-encapsulated LNA ASOs were removed by dialysis against DPBS for 72 hours at 4° C. using a MWCO of 300 kDa (Float-a-lyzer, No. G235036, Repligen, Waltham, Mass., USA).

Characterization of LNA ASO Loaded Lipid Vesicles

Lipid vesicles loaded with LNA ASO can be characterized by the following analysis's

Analysis of Purification

Removal of non-encapsulated LNA ASOs can be confirmed using agarose gel electrophoresis. 20 μL of LNA ASO loaded lipid vesicles are sonicated for three times 5 min at an amplitude of 75 to release LNA ASO cargo (encapsulated LNA ASOs). 20 μL of non-treated LNA ASO loaded lipid vesicles, 20 μL of sonicated LNA ASO loaded lipid vesicles and LNA ASO standards in DPBS are loaded into a SYBR Green labeled 2% agarose gel (No. G521802, Thermo Scientific, Waltham, Mass., USA) and nucleic acids are separated for 10 min. Gels are then imaged using an E-Gel imager (Thermo Scientific. Waltham, Mass., USA).

Quantification of LNA ASO Drug Loading Content

Drug loading content was analyzed using HPLC (Water, Milford, Mass., USA). Samples were injected on a C18 column (XBridge BEH C18 2.5 μm, 4.6×100 mm Column XP; No. 186006039, Waters, Milford, Mass., USA) and samples were run on an acetonitrile/200 mM acetate gradient at a column temperature of 50° C. LNA ASOs were detected at a wavelength of 260 nm. A calibration curve of LNA ASOs was prepared and used to quantify amount of encapsulated LNA ASOs.

In Vitro Cell Cultures

Primary Human Hepatocytes

Primary human cryopreserved hepatocytes (PHH, No. F00995, BioIVT, West Sussex, UK) were cultured in Williams Medium E (No. W1878, Sigma Aldrich, St. Louis, Mich., USA) supplemented with 10 U/mL Penicillin and Streptomycin (No. 15140122, Thermo Scientific, Waltham, Mass., USA) and 10% FBS (No. 97068-085, VWR International, Radnor, Pa., USA) at 37° C. and 5% CO₂. 4×10⁴ cells were plated on collaged coated (No. 356407, BD Biosciences, Franklin Lakes, N.J., USA) 96-well culture plates (No. 3474, Corning, Corning, N.Y., USA). Cells were allowed to adhere for 4 hours before addition of test substances.

HepG2 Cells

HepG2 human hepatocellular carcinoma cells (ATCC HB-8065) were cultured in Eagle's Minimum Essential Medium (No. M2279, Thermo Scientific, Waltham, Mass., USA) supplemented with 10% FBS (No. A3160401, Thermo Scientific, Waltham, Mass., USA) and Glutamax (No. 41090101, Thermo Scientific, Waltham, Mass., USA) at 37° C. and 5% CO₂. For knockdown experiments, cells were washed once with DPBS (No. 14190250, Thermo Scientific, Waltham, Mass., USA) and detached using 0.25% Trypsin-EDTA (No. 25200056, Thermo Scientific, Waltham, Mass., USA). Complete culture medium was added and cells were collected by centrifugation at 1300 rpm and 5 min. The supernatant was aspirated and the cell pellet was resuspended in complete culture medium. Dead cells were stained using 0.4% Trypan blue (No. T10282, Thermo Scientific, Waltham, Mass., USA) and cells were counted using a Countess II FL Automated Cell Counter (Thermo Scientific, Waltham, Mass., USA). Cells were then seeded in a 96-well plate (No. 3474, Corning, Corning, N.Y., USA) at a density of 2×10⁴ cells per well and allowed to adhere overnight before addition of test substances.

Lactate Dehydrogenase Assay

PHH cell culture supernatants were collected 48 hours after incubation with lipid vesicles and LDH release was determined using the Cytotoxicity Detection kit (No. 11644793001, Roche Diagnostics, Rotkreuz, Switzerland) according to the manufacturers' recommendations. In brief, collected cell culture supernatants were diluted and transferred to clear bottom 96-well plates (No. 3906, Corning, Corning, N.Y., USA). Cytotoxicity Detection reaction mix was freshly prepared and added to each well, plates were shaked at 1000 rpm for 1 min and incubated for 30 min at room temperature. Absorbance was measured at 490 nm using an EnSpire plate reader (Perkin Elmer, Waltham, Mass., USA). LDH concentration was determined using Precipath U (No. 10171778122, Roche Diagnostics, Rotkreuz, Switzerland) as LDH standards. DPBS treated cells were used as control. Results are expressed as mean±SD of n=4 technical replicates.

Intracellular ATP Content

Intracellular ATP content in lipid vesicle treated PHHs was determined 48 hours after incubation using Cell Titer Glo kit (No. G7571, Promega, Madison, Wis., USA) according to the manufacturers' instruction. In brief, 50 μL of Cell Titer Glo reagent was added to each well containing treated PHHs (see section 3.11) in 50 μL of complete culture medium and plates were shaked for 2 min to induce cell lysis and plates were incubated for 10 min at room temperature. 70 μL of cell lysates were transferred into a white opaque 96-well plate (No. 3601, Corning, Corning, N.Y., USA) and luminescence was collected on a EnVision plate reader (Perkin Elmer, Waltham, Mass., USA). Dilutions of ATP in complete culture medium were used as standards. DPBS treated cells were used as control. Results are expressed as mean±SD of n=4 technical replicates.

Albumin Release Assay

Albumin secretion by PHH after lipid vesicle treatment was determined 48 hours after incubation using the AlphaLISA biotin-free human serum albumin detection kit (No. AL363, Perkin Elmer, Waltham, Mass., USA) according to the manufacturers' recommendation. Results are shown as mean±SD of n=4 technical replicates.

ASO Concentrations in Tissue Homogenates Using Hybridization ELISA (hELISA)

The general method is described in Straarup et al 2010 Nucleic Acids Res, 38(20): p. 7100-11. In brief, capture detection solution (CDS) was prepared by supplementing 5×SSC buffer (No. 93017, Sigma Aldrich, St. Louis, Mich., USA) with 0.05% Tween-20 (No. P9416, Sigma Aldrich, St. Louis, Mich., USA) and adding biotinylated capture probe and digoxigenin conjugated detection probe at a final concentration of 35 nM (RTR No. 35148-3 and No. 31443-7, Roche Innovation Center Copenhagen, Horsholm, Denmark). Streptavidin coated 96-well plates (No. 436014, Thermo Scientific, Waltham, Mass., USA) were washed three times with 5×SSC-T buffer and 100 μL of tissue homogenates (diluted 1:10 in CDS) or RTR17293 standards in CDS were added. Samples were incubated for 1 hour at room temperature under constant agitation. Plates were washed three times with 2×SSCT buffer. Wells were incubated with alkaline phosphatase conjugated anti-digoxigenin antibody (No. 11093274910, Roche Diagnostics, Rotkreuz, Switzerland) diluted 1:3′000 in DPBS supplemented with 0.05% Tween-20 for 1 hour at room temperature under constant agitation. Plates were washed three times with 2×SSCT buffer. Blue Phos substrate (No. 55-88-02, Seracare Life Sciences, Milford, Mass., USA) was mixed according to manufacturer's instructions and 100 μL of substrate were added to each well. Absorbance was measured at a wavelength of 600 nm using GlowMax Discoverer plate reader (Promega, Madison, Wis., USA) and unknown concentrations were determined by preparing a sigmoidal (4PL) standard curve of standards using GraphPad Prism Version 6.07 (GraphPad Software, La Jolla, Calif., USA). Results are expressed as mean±SD of n=4 replicates.

### Example 1: Preparation of Lipid Vesicles

The present example describes the components of and the preparation of lipid vesicles tested in the present invention.

TABLE 2
Lipids/Components used:

| Lipid component | MW [g* mol⁻¹] | mol % |
|---|---|---|
| Cholesterol (Sigma Aldrich, No C8667) | 387 | 14 |
| Milk sphingomyelin (SM) (Avanti Polar Lipids | 711 | 5 |
| No 860063P) | | |
| Brain SM (Avanti Polar Lipids, No. 860062P) | | |
| Egg SM (Avanti Polar Lipids, No. 860061P) | | |
| Egg phosphatidylcholine (PC) (Avanti Polar | 770 | 29 |
| Lipids, No 840051P) | | |
| DSPC (Avanti Polar Lipids, No 850365P) | | |
| DLPC (Avanti Polar Lipids, No 850335P) | | |
| DOPC (Avanti Polar Lipids, No 850311P) | | |
| L-alpha phosphatidylethanolamine (PE) (P1223) | 744 | 19 |
| L-alpha phosphatidylserine (PS) (P0474) | 792 | 14 |
| Dipalmitin (DAG) (D2636) | 569 | 5 |
| Glyceryl Tristearate (TAG) (T5016) | 891 | 5 |
| Liver phosphatidylinositol (PI) (840042P) | 902 | 5 |
| Bis(monooleoylglycero)phosphate (S,R Isomer) | 792 | 5 |
| (18:1 BMP (S,R)) (857133P)(LBPA) | | |

Product numbers are indicated in brackets.

(a) Thin Film Rehydration

The lipid vesicles (liposomes) listed in table 3 below were prepared using the thin film rehydration method. Lipids were dissolved in chloroform/methanol (2:1 v/v) and mixed at indicated molar ratios. The lipids were transferred to a round bottom flask and the solvents were removed on a rotavapor at 65° C. and <20 mbar. Dry lipid films were rehydrated with phosphate buffered solution (PBS) using 1.5 g of 3 mm glass beads at 65° C. at a final lipid concentration of 10 mM. Large multilammelar vesicles (LUVs) were subjected to 5 freeze-thaw cycles (dry ice and 65° C.) to break multilammellar structures. Subsequently, vesicle size was reduced by extrusion using a hand extruder (Avanti Polar Lipids) with 200 nm pore size for 11-21 times at 65° C. Liposomes where stored at 4° C. for <1 week until further analysis. The respective amounts of the components of the prepared vesicles are given in Table 3 in [mol].

(b) Microfluidics

Selected lipid vesicles can alternatively be prepared using microfluidics. Lipids are dissolved in an appropriate water miscible solvent such as ethanol, methanol, DMSO, DMF, or acetone. Liposomes are then prepared by mixing lipids with aqueous buffer using the NanoAssemblr (Precision Nanosystems, Vancouver BC, Canada). Flow rate ratio (FRR) and total flow rate (TFR) can be adjusted accordingly. Solvents are then removed by dialysis using a molecular weight cut-off of 300 kDa against PBS.

(c) Probe Sonification

Lipids of formulation F1 (table 3) at a total lipid concentration of 10 mM were dispersed in 20 mM citrate buffer (pH 5.0). Small, homogeneous lipid vesicles were then prepared by two cycles of sonication (30 and 20 m2) at an amplitude of 10 using a probe sonication (Q700, Qsonica L.L.C., Newtown, Conn., USA). Small lipid vesicles were then sterilly filtered using a 0.22 μm syringe filter (Merck KGaA, Darmstadt, Germany) and stored at 400.

TABLE 3
Lipid compositions of prepared vesicles. The lipids used are listed in table 2

| Vesicle | | | | | | | | | |
|---|---|---|---|---|---|---|---|---|---|
| No | Chol [mol] | SM¹ [mol] | PC⁴ [mol] | PE [mol] | PS [mol] | DAG [mol] | TAG1 [mol] | PI [mol] | LBPA1 [mol] |
| F1 | 14.3 | 4.8 | 28.6 | 19 | 14.3 | 4.8 | 4.8 | 4.8 | 4.8 |
| F2 | 0 | 5.6 | 33.3 | 22.2 | 16.7 | 5.6 | 5.6 | 5.6 | 5.6 |
| F3 | 15 | 0 | 30 | 20 | 15 | 5 | 5 | 5 | 5 |
| F4 | 20 | 6.7 | 0 | 26.7 | 20 | 6.7 | 6.7 | 6.7 | 6.7 |
| F5 | 17.6 | 5.9 | 35.3 | 0 | 17.6 | 5.9 | 5.9 | 5.9 | 5.9 |
| F6 | 16.7 | 5.6 | 33.3 | 22.2 | 0 | 5.6 | 5.6 | 5.6 | 5.6 |
| F7 | 15 | 5 | 30 | 20 | 15 | 0 | 5 | 5 | 5 |
| F8 | 15 | 5 | 30 | 20 | 15 | 5 | 0 | 5 | 5 |
| F9 | 15 | 5 | 30 | 20 | 15 | 5 | 5 | 0 | 5 |
| F10 | 15 | 5 | 30 | 20 | 15 | 5 | 5 | 5 | 0 |
| F11 | 30 | 10 | 60 | 0 | 0 | 0 | 0 | 0 | 0 |
| F12 | 17.6 | 5.9 | 35.3 | 23.5 | 17.6 | 0 | 0 | 0 | 0 |
| F13 | 16.7 | 5.6 | 33.3 | 22.2 | 16.7 | 0 | 0 | 0 | 5.6 |
| F14 | 33.3 | 11.1 | 0 | 44.4 | 0 | 0 | 0 | 0 | 11.1 |
| F15 | 18.8 | 0 | 37.5 | 0 | 18.8 | 6.3 | 6.3 | 6.3 | 6.3 |
| F16 | 75 | 25 | 0 | 0 | 0 | 0 | 0 | 0 | 0 |
| F17 | 37.5 | 12.5 | 0 | 50 | 0 | 0 | 0 | 0 | 0 |
| F18 | 30 | 17.5 | 21 | 17.5 | 14 | 0 | 0 | 0 | 0 |
| F19 | 17.6 | 5.9 | 35.3⁵ | 23.5 | 17.6 | 0 | 0 | 0 | 0 |
| F20 | 17.6 | 5.9 | 35.3⁶ | 23.5 | 17.6 | 0 | 0 | 0 | 0 |
| F21 | 17.6 | 5.9 | 35.3⁷ | 23.5 | 17.6 | 0 | 0 | 0 | 0 |
| F22 | 17.6 | 5.9² | 35.3 | 23.5 | 17.6 | 0 | 0 | 0 | 0 |
| F23 | 17.6 | 5.9³ | 35.3 | 23.5 | 17.6 | 0 | 0 | 0 | 0 |
| F24 | 5.9 | 23.5 | 23.5 | 23.5 | 23.5 | 0 | 0 | 0 | 0 |
| F25 | 50 | 12.5 | 12.5 | 12.5 | 12.5 | 0 | 0 | 0 | 0 |
| F26 | 30.2 | 7.5 | 1.9 | 30.2 | 30.2 | 0 | 0 | 0 | 0 |
| F27 | 4.3 | 17.4 | 69.6 | 4.3 | 4.3 | 0 | 0 | 0 | 0 |
| F28 | 42.1 | 2.6 | 10.5 | 2.6 | 42.1 | 0 | 0 | 0 | 0 |
| F29 | 2.6 | 42.1 | 10.5 | 42.1 | 2.6 | 0 | 0 | 0 | 0 |
| F30 | 42.1 | 42.1 | 2.6 | 10.5 | 2.6 | 0 | 0 | 0 | 0 |
| F31 | 2.6 | 2.6 | 42.1 | 10.5 | 42.1 | 0 | 0 | 0 | 0 |
| F32 | 30.2 | 30.2 | 30.2 | 1.9 | 7.5 | 0 | 0 | 0 | 0 |
| F33 | 4.3 | 4.3 | 4.3 | 69.6 | 17.4 | 0 | 0 | 0 | 0 |
| F34 | 20 | 20 | 20 | 20 | 20 | 0 | 0 | 0 | 0 |

¹Milk SM unless indicated otherwise
²Brain SM
³Egg SM
⁴Egg PC unless indicated otherwise
⁵DSPC
⁶DLPC
⁷DOPC

Stability Testing

The stability of the lipid vesicles in simulated gastric (SGF: NaCl 34 mM, HCl 0.83 M, 0.1% Triton X-100 (pH 1.2)) and fasted-state intestinal fluids (FaSSIF: NaH₂PO₄ 28.6 mM, Taurocholic acid sodium salt 3 mM, Lecithin 0.75. NaCl 105.8 mM (pH 6.5)) was tested overtime by measuring the change in Dh by DLS, as described in the Materials and Method section above.

The lipid vesicles were mixed with SGF or FaSSIF at a final lipid concentration of 1 mM and Dh was measured at 20° C. at indicated time points. Data are shown as mean of n=1 experiment.

TABLE 4
Results of stability testing, in the table the size (Dh) is given in nm, as well as the
change in size after 5 h incubation compared to the size a 0 h.

| | SGF | | | | FaSSIF | | | |
|---|---|---|---|---|---|---|---|---|
| Time [h] | 0 | 3 | 5 | % Dh change | 0 | 3 | 5 | % Dh change |
| F1 | 81.0 | 128.6 | 123.6 | 53 | 81.0 | 94.1 | 98.0 | 21 |
| F2 | 108.9 | 248.5 | 190.4 | 75 | 108.9 | 139.9 | 145.1 | 33 |
| F3 | 75.3 | 111.8 | 139.6 | 85 | 75.3 | 81.6 | 82.5 | 10 |
| F4 | 113.2 | 669.7 | 1768.4 | 1462 | 113.2 | 132.7 | 147.6 | 30 |
| F5 | 134.7 | 0.0 | 3840.4 | 2751 | 134.7 | 384.7 | 274.7 | 104 |
| F6 | 88.7 | 112.6 | 213.5 | 141 | 88.7 | 183.5 | 96.4 | 9 |
| F7 | 170.1 | 259.0 | 364.9 | 115 | 170.1 | 241.0 | 293.7 | 73 |
| F8 | 134.7 | 179.7 | 425.5 | 216 | 134.7 | 128.7 | 131.2 | 3 |
| F9 | 171.1 | 351.4 | 499.8 | 192 | 171.1 | 231.9 | 263.8 | 54 |
| F10 | 155.1 | 283.7 | 327.1 | 111 | 155.1 | 499.3 | 392.0 | 153 |
| F11 | 182.4 | 813.0 | 385.7 | 111 | 182.4 | 226.1 | 270.2 | 48 |
| F12 | 142.3 | 114.1 | 119.2 | 16 | 142.3 | 129.8 | 123.0 | 14 |
| F13 | 136.8 | 118.6 | 125.5 | 8 | 136.8 | 128.1 | 124.1 | 9 |
| F14 | 108.0 | 136.0 | 134.3 | 24 | 108.0 | 105.7 | 105.7 | 2 |
| F15 | 85.5 | 278.5 | 176.1 | 106 | 85.5 | 128.1 | 129.3 | 51 |
| F16 | 1050.0 | 1272.0 | 847.1 | 19 | 1050.7 | 1160.6 | 890.4 | 15 |
| F17 | 110.7 | 117.7 | 161.5 | 46 | 110.7 | 107.4 | 107.5 | 3 |
| F18 | 150.2 | 202.9 | 271.4 | 81 | 150.2 | 143.8 | 138.6 | 8 |
| F19 | 221.1 | 384.1 | 387.4 | 75 | 221.1 | 393.4 | 426.0 | 93 |
| F20 | 163.7 | 252.2 | 222.2 | 36 | 163.7 | 130.3 | 135.4 | 17 |
| F21 | 150.4 | 125.9 | 124.0 | 18 | 150.4 | 139.0 | 134.2 | 11 |
| F22 | 160.4 | 127.0 | 131.5 | 18 | 160.4 | 149.0 | 144.5 | 10 |
| F23 | 149.8 | 123.8 | 124.0 | 17 | 149.8 | 143.9 | 139.7 | 7 |
| F24 | 240.0 | 144.0 | 147.0 | 39 | 240.0 | 177.0 | 177.0 | 26 |
| F25 | 151.0 | 160.0 | 162.0 | 7 | 151.0 | 146.0 | 147.0 | 3 |
| F26 | 115.0 | 56.7 | 53.7 | 53 | 115.0 | 113.0 | 112.0 | 3 |
| F27 | 161.0 | 36.2 | 29.6 | 82 | 161.0 | 166.0 | 54.7 | 66 |
| F28 | 251.0 | 180.0 | 176.0 | 30 | 251.0 | 263.0 | 240.0 | 4 |
| F29 | 339.0 | 19.4 | 20.9 | 94 | 339.0 | 25.7 | 83.9 | 75 |
| F30 | 216.0 | 219.0 | 226.0 | 5 | 216.0 | 287.0 | 292.0 | 35 |
| F31 | 141.0 | 100.0 | 95.2 | 32 | 141.0 | 49.1 | 41.8 | 70 |
| F32 | 723.0 | 706.0 | 788.0 | 9 | 723.0 | 1200.0 | 1150.0 | 59 |
| F33 | 127.0 | 146.0 | 144.0 | 13 | 127.0 | 124.0 | 122.0 | 4 |
| F34 | 216.0 | 271.0 | 270.0 | 25 | 216.0 | 293.0 | 298.0 | 38 |

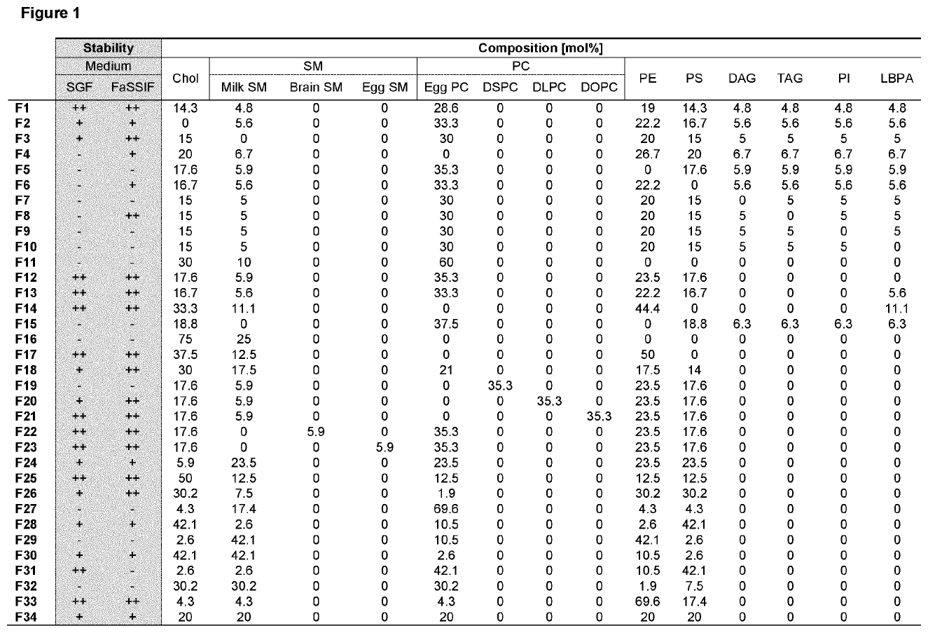

Based on the data in table 4 the following lipid vesicle compositions F1, F3, F12, F13, F14, F17, F18, F20, F21, F22, F23, F24, F25, F26, F28, F30, F33, F34 have been characterized as stable and suitable for oral delivery. In particular lipid vesicle compositions F1, F12, F13, F14, F17, F21, F22, F23, F25, F33, are considered particular suitable for oral delivery. A summary of the lipid components of the lipid vesicles and the stability is shown in FIG. 1.

### Example 2 In Vitro Evaluation of Lipid Vesicle F1

It was investigated whether the purified lipid vesicle F1 from example 1 was tolerated in an in vitro cell culture.

Primary human cryopreserved hepatocytes were cultivated as described in the Materials and Methods section. The lipid vesicles were diluted in FBS free culture medium to reach final assay concentrations of 0.04 and 0.4 mg/mL based on total lipid concentration determined by HPLC analysis. The lipid vesicles were added to wells and PHHs were incubated for 24 hours at 37° C. Medium was exchanged with complete culture medium and PHHs were incubated for another 24 hours at 37° C.

The tolerability was assessed using lactate dehydrogenase assay, intracellular ATP content and albumin release assay, all described in the Materials and Methods section.

The results are shown in table 5, and clearly indicate that the lipid vesicle of F1 is well tolerated by the PPH cells.

TABLE 5
In vitro toxicity evaluation on cryopreserved primary
human hepatocytes (PHHs).
Results are shown as mean ± SD of n = 4 technical replicates.

| | DPBS | lipid vesicle F1 | |
|---|---|---|---|
| Concentration [mg lipid/mL] | — | 0.04 | 0.4 |
| Lactate dehydrogenase | 206 ± 18 | 201 ± 8 | 205 ± 22 |
| concentration in culture | | | |
| medium supernatant [mU/mL] | | | |
| Intracellular ATP content | 172 ± 5 | 174 ± 2 | 160 ± 6 |
| [pmol/well] | | | |
| Albumin concentration in culture | 0.47 ± 0.03 | 0.49 ± 0.04 | 0.49 ± 0.06 |
| medium supematant [μg/mL] | | | |

### Example 3 Loading of LNA ASO into Lipid Vesicle F1

The lipid vesicle F1 from example 1 was loaded with ASO1 by rehydrating the thin lipid film in the presence of LNA-ASO as described in the Materials and Methods section.

The physico-chemical properties as well as the LNA ASO content in the loaded lipid vesicle was analyzed as describe in the Materials and Methods section. The results of lipid vesicle loaded with ASO1 are shown in table 6 below. The LNA ASO loaded lipid vesicle was stable over 2 months at 4° C. as shown by constant size and PDI

TABLE 6
Physico-chemical characterization of biomimetic lipid vesicles.
Results are shown as mean ± SD of n = 3 experiments.

| | unloaded | |
|---|---|---|
| | lipid vesicle | ASO1 loaded lipid |
| | F1 | vesicle F1 |
| Hydrodynamic diameter (Dh) [nm] | 115.6 ± 18.3 | 144.1 ± 28.4 |
| Polydispersity index (PDI) | 0.131 ± 0.020 | 0.096 ± 0.025 |
| Zeta potential [mV]¹ | −40.3 ± 3.4 | −42.7 ± 15.7 |
| LNA ASO loading content [% w/w]² | — | 3.4 ± 1.0 |

¹Determined in 0.1 DPBS (0.81 mM phosphate, 13.7 mM NaCl)
²LNA ASO content per mg of lipid

### Example 4 In Vitro Evaluation of LNA ASO-Loaded Lipid Vesicle F1 in Hep2G Cells

Lipid vesicle F1 loaded with ASO1 as described in example 3 was tested in HepG2 cells to assess whether is tolerated in an in vitro cell culture and whether a reduction of the target can be achieved.

The HepG2 cells were cultivated as described in the Materials and Method section. The cells were treated with ASO1 in DPBS (No. 14190250, Thermo Scientific, Waltham, Mass., USA) or ASO1 encapsulated in lipid vesicles with the following LNA-ASO concentration 10, 50, 100, 500, 1′000, and 5′000 nM. Cells were incubated for 24 hours at 37° C. Then, the medium was aspirated, cells were washed once with DPBS and incubated with fresh complete culture medium for another 24 hours. 48 hours after incubation, cells were washed twice with DPBS.

RNA was isolated using the PureLink Pro 96 total RNA Purification kit (No. 12173011A, Thermo Scientific, Waltham, Mass., USA). RNA expression was analyzed using the LightCycler Multiplex RNA Virus Master kit (No. 07083173001, Roche Diagnostics, Rotkreuz, Switzerland) on a LightCycler 480 instrument. Malat-1 lncRNA (Hs00273907_s1) and GAPDH (Hs02786624_g1) primers were ordered from Thermo Scientific (Waltham, Mass., USA). Malat-1 lncRNA expression was analyzed by ΔΔct method against GAPDH. Results are expressed as mean fold-change ±SD of n=4 technical replicates.

The results are shown in table 7, and indicate that malat-1 target knockdown can be achieved in vitro with the LNA-ASO-loaded lipid vesicles to the same level as the DPBS formulated LNA-ASO.

TABLE 7
In vitro evaluation of LNA-ASO loaded lipid vesicles in HEP2G cells.
Results are shown as percentage of control mean ± SD
of n = 4 technical replicates.

| Concentration ASO1 [nM] | ASO1 in DPBS | ASO1 in lipid vesicle F1 |
|---|---|---|
| 10 | 73 ± 11 | 80 ± 11 |
| 50 | 88.2 ± 13 | 72 ± 8 |
| 100 | 73 ± 5 | 61 ± 13 |
| 500 | 60 ± 2 | 55 ± 15 |
| 1000 | 55 ± 10 | 59 ± 9 |
| 5000 | 50 ± 8 | 53 ± 10 |

### Example 5 In Vivo Evaluation of LNA-ASO-Loaded Lipid Vesicle F1

The biodistribution and ability to achieve target reduction of the LNA-ASO-loaded lipid vesicle's was asses in vivo in mice using either intravenous injection or oral administration.

All animal experiments were performed according to Swiss animal protection legislation and in accordance with international guidelines and good practices.

C57BL6 mice were administered with ASO1 in DPBS orASO1 encapsulated in lipid vesicle F1 as described in example 3. The administration protocol is summarized in the table 8 below

TABLE 8
in vivo administration protocol

| Group | | | |
|---|---|---|---|
| (No of | | | Dose ASO1 |
| animals) | Treatment | Application route | [mg/kg BW] |
| 1 (2) | Vehicle control (DPBS) | i.v. bolus injection | — |
| 2 (4) | ASO1 in lipid vesicle F1 | i.v. bolus injection | 0.5 |
| 3 (4) | ASO1 in DPBS | i.v. bolus injection | 0.425 |
| 4 (4) | ASO1 in lipid vesicle F1 | Oral gavage | 1.0 |

Mice were sacrificed 3 days after treatment, organs were collected and rinsed in 0.9% NaCl. Spleens were dissected in two pieces and one half was placed in RPMI-1640 culture medium (No. 11875093, Thermo Scientific, Waltham, Mass., USA) at 4° C. for immediate T-cell isolation (see below). All other organs were stored in RNAlater solution (No. AM7021, Thermo Scientific, Waltham, Mass., USA) at −80° C. until further processing. Organs were placed in 3× volumes of QIAzol lysis reagent (No. 79306, Qiagen, Hilden, Germany), transferred into 2 mL or 5 mL Precellys tubes (No. KT03961-1-002.2 or No. KT03961-1-302.7, Bertin Instruments, Montigny-le-Bretonneux, France) containing ceramic beads, and homogenized using a Precellys evolution homogenizer (Bertin Instruments, Montigny-le-Bretonneux, France). ASO1 concentrations in tissue homogenates were determined using hybridization ELISA (hELISA) as described in the Materials and Method section. The biodistribution of ASO 1 is show in in table 9, clearly indicating distribution of the LNA_ASO-loaded lipid vesicles to several tissues both when given I.V. and by oral gavage. With I.V. administration appearing to provide the largest oligonucleotide concentration.

TABLE 9
In vivo biodistribution evaluation of ASO1. Concentrations of ASO1 in
the indicated tissues is in nM and shown as mean ± SD
of n = 4 replicates.

| | | ASO1 in lipid | ASO1 in lipid |
|---|---|---|---|
| Treatment | ASO1 in DPBS | vesicle F1 | vesicle F1 |
| Administration route | i.v. injection | i.v. injection | p.o. gavage |
| Liver | 6.533 ± 3.635 | 21.005 ± 10.283 | 2.130 ± 0.618 |
| Kidney left | 14.098 ± 0.660 | 10.696 ± 1.227 | 0.822 ± 0.439 |
| Kidney right | 14.682 ± 1.218 | 11.378 ± 1.028 | 1.586 ± 1.202 |
| Small intestine | 1.822 ± 0.353 | 2.934 | 1.629 ± 0.850 |
| Brain | n.d. | n.d. | n.d. |
| Spleen | 3.623 ± 0.868 | 13.147 ± 5.490 | 3.077 |
| T-cells | n.d. | n.d. | n.d. |

nd = not detectable by hELISA

The knockdown of target RNA (Malat-1 mRNA) was also analyzed in the collected tissues by RT-PCR. In brief total RNA was isolated from tissue homogenates using the miRNeasy Mini Kit (No. 217004, Qiagen, Hilden, Germany) according to the manufacturers' recommendations. Total RNA was quantified using the Quant-iT RiboGreen RNA Assay Kit (R11490, Thermo Scientific, Waltham, Mass., USA). RNA expression was analyzed using the LightCycler Multiplex RNA Virus Master kit (No. 07083173001, Roche Diagnostics, Rotkreuz, Switzerland) on a LightCycler 480 instrument (Roche Diagnostics, Rotkreuz, Switzerland). Malat-1 lncRNA (Mm01227912_s1) and GAPDH (Mm99999915_g1) primers were ordered from Thermo Scientific (Waltham, Mass., USA). Malat-1 mRNA expression was analyzed by ΔΔct method against GAPDH/normalized to total RNA content. The results are shown in table 10 and are expressed as mean fold-change ±SD of n=4 replicates. The results show that target reduction can be achieved in liver, brain, spleen and T-cells. The LNA ASO-loaded lipid vesicle's administered by oral gavage seem to result in a very efficient target knock down in the spleen. Following oral administration some knock down is also observed in the brain and T-cells although it does not exceed that achieved by I.V. administration of ASO in DPS or ASO in lipid vesicles. Target knock down in the kidney seems to be eliminated when ASO is administered orally in lipid vesicles.

TABLE 10
In vivo target knock-down efficiency of ASO1 LNA ON. ASO1
formulated in DPBS or encapsulated in lipid vesicle (F1) was
administered in mice via intravenous injection (0.5 mg/kg) or oral gavage
(1 mg/kg). Knockdown of target RNA (Malat-1) was analyzed by rt-
qPCR normalized to the total RNA content. Expression values are shown
as fold-change of control (DPBS treated animals) ± SD of n = 4 replicates.

| Treatment | DPBS | ASO1 | ASO1 in lipid | ASO1 in lipid |
|---|---|---|---|---|
| Administration | i.v. | in DPBS | vesicles (F1) | vesicles (F1) |
| route | injection | i.v. injection | i.v. injection | p.o. gavage |
| Liver | 100 ± 34 | 38 ± 16 | 61 ± 15 | 54 ± 6 |
| Kidney left | 100 ± 22 | 72 ± 13 | 44 ± 3 | 46 ± 5 |
| Kidney right | 100 ± 37 | 47 ± 3 | 30 ± 7 | 43 ± 4 |
| Small intestine | 100 ± 12 | 94 ± 47 | 69 ± 24 | 99 ± 9 |
| Brain | 100 ± 12 | 65 ± 17 | 43 ± 7 | 46 ± 14 |
| Spleen | 100 ± 0 | 29 ± 29 | 10 ± 11 | 3 ± 3 |
| T-cells | 100 ± 12 | 80 ± 12 | 63 ± 11 | 44 ± 11 |

1.-21. (canceled)

22. A lipid vesicle carrying a nucleic acid molecule for use as a medicament, wherein the lipid vesicle has a hydrodynamic diameter Dh of less than 300 nm, measured according to DLS, preferably a diameter in the range from 50 to 300 nm, wherein said lipid vesicle 5 is administered orally, wherein said nucleic acid molecule is for delivery to one or more of the target tissues selected from the group consisting of the central nervous system, spleen, gastrointestinal tract, liver and T-cells.
